SEQUENCE LISTING (40 sequences)

SEQ ID NO: 1 - protein, 13 aa, synthetic construct
  SITE 1: note = Acetylated Cysteine
CEPWWTLPENFHS

SEQ ID NO: 2 - protein, 16 aa, synthetic construct
  SITE 1: note = Acetylated Cysteine
CDDLVTSVSLPPYTGD

SEQ ID NO: 3 - DNA, 432 bp, synthetic construct
  source 1..432: mol_type = other DNA
atgggatggagctgtatcatgctcttcttggcagcaacagctacaggtgtccactcccaggtccaactgcagcagcctggggctgagtttgtgaagcctggggcttcagtgaagctgtcctgcaaggcttctgactacaccttcaccaactactggatgcactgggtgaaacagaggcctggacgaggccttgagtggattggaaggattgatcctaacagtggtggtactaagtataaggagaagttcaagagaagggtcacactgactgtagacaagccctccagcacagtctacatgcagctcaacagcctgacatctgaggactctgcggtctatttctgtgcaagagaaagggctatttcctacggtagtagccccctaaattctctggactactggggtcaaggaacctcagtcaccgtctcctca

SEQ ID NO: 4 - protein, 144 aa, synthetic construct
MGWSCIMLFLAATATGVHSQVQLQQPGAEFVKPGASVKLSCKASDYTFTNYWMHWVKQRPGRGLEWIGRIDPNSGGTKYKEKFKRRVTLTVDKPSSTVYMQLNSLTSEDSAVYFCARERAISYGSSPLNSLDYWGQGTSVTVSS

SEQ ID NO: 5 - DNA, 399 bp, synthetic construct
  source 1..399: mol_type = other DNA
atggattcacaggcccaggttcttatgttactgctgctatgggtatctggtacctgtggggacattgtgatgtcacagtctccatcctccctagctgtgtcagttggagagaaggttactatgagctgcaagtccagtcagagccttttatatagtagcaatcaaaagaactacttggcctggtaccagcagaaaccagggcagtctcctaaactgatgatttactgggcatccactagggaatctggggtccctgatcgcttcacaggcagtggatctgggacagatttcactctcaccatcagcagtgtgaaggctgaagacctggcagtttattactgtcagcaatattatagctatccgtacacgttcggaggggggaccaagctggaaataaaa

SEQ ID NO: 6 - protein, 133 aa, synthetic construct
MDSQAQVLMLLLLWVSGTCGDIVMSQSPSSLAVSVGEKVTMSCKSSQSLLYSSNQKNYLAWYQQKPGQSPKLMIYWASTRESGVPDRFTGSGSGTDFTLTISSVKAEDLAVYYCQQYYSYPYTFGGGTKLEIK

SEQ ID NO: 7 - DNA, 402 bp, synthetic construct
  source 1..402: mol_type = other DNA
atgggatggagctggatctttctctttctcctgtcaggaactgcaggtgtcctctctgaggtccagctgcaacaatctggacctgagctggtgaagcctggggcctcagtgaagatatcctgtaaggcttctggatacacgttcactgaatactacatgaactgggtgaagcagagccctggaaagagccttgagtggattggagatattaatcctaacaatggtggtacttactataatcagatgttcaagggcaaggccacattgactgtagacaagtcctccaccacagcctacatggagctccgcagcctgacatctgaggactctgcagtctattactgcgcaagaaaagtctcctctccttactggggccaagggactctggtcactgtctctgca

SEQ ID NO: 8 - protein, 134 aa, synthetic construct
MGWSWIFLFLLSGTAGVLSEVQLQQSGPELVKPGASVKISCKASGYTFTEYYMNWVKQSPGKSLEWIGDINPNNGGTYYNQMFKGKATLTVDKSSTTAYMELRSLTSEDSAVYYCARKVSSPYWGQGTLVTVSA

SEQ ID NO: 9 - DNA, 396 bp, synthetic construct
  source 1..396: mol_type = other DNA
atgaggtgcctagctgagttcctggggctgcttgtgctctggatccctggagtcattggggatattgtgatgactcaggctgcaccctctgtatttgtcactcctggagagtcagtatccatctcctgcaggtctactaagagtctcctgcatagtaatggcaacacttatttgtattggttcctgcagaggccaggccagtctcctcaggtcctgatatatcggatgtccaaccttgcctcaggagtcccagacaggttcagtggcagtgggtcaggaactgctttcacactgagaatcagtagagtggaggctgaggatgtgggtgtttattactgtatgcaacatctagaatatcctttcacgttcggctcggggacaaagttggaaataaaa

SEQ ID NO: 10 - protein, 132 aa, synthetic construct
MRCLAEFLGLLVLWIPGVIGDIVMTQAAPSVFVTPGESVSISCRSTKSLLHSNGNTYLYWFLQRPGQSPQVLIYRMSNLASGVPDRFSGSGSGTAFTLRISRVEAEDVGVYYCMQHLEYPFTFGSGTKLEIK

SEQ ID NO: 11 - protein, 14 aa, synthetic construct
  SITE 1: note = Cysteine(Carbamidomethyl)
CIELHSHTLIQLER

SEQ ID NO: 12 - protein, 10 aa, synthetic construct
VTVVGPPMAK

SEQ ID NO: 13 - protein, 14 aa, synthetic construct
  SITE 1: note = Cysteine(Carbamidomethyl)
CIELHSHTLIQLER

SEQ ID NO: 14 - protein, 9 aa, synthetic construct
  SITE 1: note = Cysteine(Carbamidomethyl)
CFTATGQTR

SEQ ID NO: 15 - protein, 10 aa, synthetic construct
VTVVGPPMAK

SEQ ID NO: 16 - protein, 21 aa, synthetic construct
SQPLTNDDLVTSVSLPPYTGD

SEQ ID NO: 17 - protein, 7 aa, synthetic construct
DYTFTNY

SEQ ID NO: 18 - protein, 6 aa, synthetic construct
DPNSGG

SEQ ID NO: 19 - protein, 14 aa, synthetic construct
RAISYGSSPLNSLD

SEQ ID NO: 20 - protein, 13 aa, synthetic construct
SQSLLYSSNQKNY

SEQ ID NO: 21 - skipped

SEQ ID NO: 22 - protein, 6 aa, synthetic construct
YYSYPY

SEQ ID NO: 23 - protein, 7 aa, synthetic construct
GYTFTEY

SEQ ID NO: 24 - protein, 7 aa, synthetic construct
INPNNGG

SEQ ID NO: 25 - protein, 4 aa, synthetic construct
VSSP

SEQ ID NO: 26 - protein, 12 aa, synthetic construct
TKSLLHSNGNTY

SEQ ID NO: 27 - skipped

SEQ ID NO: 28 - protein, 6 aa, synthetic construct
HLEYPF

SEQ ID NO: 29 - protein, 5 aa, synthetic construct
NYWMH

SEQ ID NO: 30 - protein, 17 aa, synthetic construct
RIDPNSGGTKYKEKFKR

SEQ ID NO: 31 - protein, 16 aa, synthetic construct
ERAISYGSSPLNSLDY

SEQ ID NO: 32 - protein, 17 aa, synthetic construct
KSSQSLLYSSNQKNYLA

SEQ ID NO: 33 - protein, 7 aa, synthetic construct
WASTRES

SEQ ID NO: 34 - protein, 8 aa, synthetic construct
QQYYSYPY

SEQ ID NO: 35 - protein, 5 aa, synthetic construct
EYYMN

SEQ ID NO: 36 - protein, 17 aa, synthetic construct
DINPNNGGTYYNQMFKG

SEQ ID NO: 37 - protein, 6 aa, synthetic construct
KVSSPY

SEQ ID NO: 38 - protein, 16 aa, synthetic construct
RSTKSLLHSNGNTYLY

SEQ ID NO: 39 - protein, 7 aa, synthetic construct
RMSNLAS

SEQ ID NO: 40 - protein, 9 aa, synthetic construct
MQHLEYPFT

(19) United States
(12) Patent Application Publication
(10) Pub. No.: US 2026/0153519 A1
(43) Pub. Date: Jun. 4, 2026

(54) NOVEL BIOMARKER FOR DETERMINING DISEASE ACTIVITY OR PROGNOSIS OF FACIOSCAPULOHUMERAL MUSCULAR DYSTROPHY

(71) Applicants: Avidity Biosciences, Inc., San Diego, CA (US); Fred Hutchinson Cancer Center, Seattle, WA (US)

(72) Inventors: David Anthony CANTON, Poway, CA (US); Xueqing LIU, San Diego, CA (US); Yiming ZHU, Belmont, CA (US); Stephen TAPSCOTT, Seattle, WA (US); Sean BENNETT, Seattle, WA (US)

(21) Appl. No.: 19/373,495

(22) Filed: Oct. 29, 2025

Related U.S. Application Data
(60) Provisional application No. 63/714,086, filed on Oct. 30, 2024.

(51) Int. Cl. G01N 33/68; G01N 33/53

(52) U.S. Cl. CPC G01N 33/6893; G01N 33/5308; G01N 33/6848; G01N 2800/2878

(57) ABSTRACT

Disclosed herein is a novel biomarker and methods for determining the disease activity and prognosis of facioscapulohumeral muscular dystrophy (FSHD) using the novel biomarker.

### CROSS REFERENCE

This application claims the benefit of U.S. Provisional Application No. 63/714,086 filed Oct. 30, 2024, which is incorporated herein by reference in its entirety.

### STATEMENT OF GOVERNMENT SUPPORT

This invention was made with government support under AR045203 awarded by the National Institutes of Health. The government has certain rights in the invention.

### REFERENCE TO A SEQUENCE LISTING XML

This application contains a Sequence Listing which has been submitted electronically in XML format. The Sequence Listing XML is incorporated herein by reference. Said XML file, created on Nov. 19, 2025, is named 45532-789_201_SL.xml and is 38,033 bytes in size.

### BACKGROUND OF THE DISCLOSURE

Muscle dystrophy causes the loss of muscle mass or the progressive weakening and degeneration of muscles, such as skeletal or voluntary muscles that controls movement, cardiac muscles, and smooth muscles. Various pathophysiological conditions including disuse, starvation, cancer, diabetes, and renal failure, or treatment with glucocorticoids result in muscle atrophy and loss of strength. The phenotypical effects of muscle atrophy are induced by various molecular events, including inhibition of muscle protein synthesis, enhanced turnover of muscle proteins, abnormal regulation of satellite cells differentiation, and abnormal conversion of muscle fiber types.

Facioscapulohumeral muscular dystrophy (FSHD) is a rare, progressive and disabling disease for which there are no approved treatments. FSHD is one of the most common forms of muscular dystrophy and affects both sexes equally, with onset at any time of life but typically in teens and young adults. FSHD is characterized by progressive skeletal muscle loss that can initially cause weakness in muscles in the face, shoulders, arms and trunk and progresses to weakness in muscles in lower extremities, the pelvic girdle, abdominal and back muscles.

Skeletal muscle weakness results in significant physical limitations, including progressive loss of facial muscles that can cause an inability to smile or communicate, difficulty using arms for activities of daily living and difficulty getting out of bed, with many patients ultimately becoming dependent upon the use of a wheelchair for daily mobility activities. The majority of patients with FSHD also report experiencing chronic pain, anxiety, and depression.

FSHD is caused by aberrant expression of a gene, double homeobox 4 (DUX4), in skeletal muscle resulting in the inappropriate presence of DUX4 protein. Disease activity correlates with the level of expression of DUX4 and DUX4 regulated genes. Gene suppression by RNA-induced gene silencing provides several levels of control: transcription inactivation, small interfering RNA (siRNA)-induced mRNA degradation, and siRNA-induced transcriptional attenuation. In some instances, RNA interference (RNAi) provides long lasting effect over multiple cell divisions and in non-dividing cells. As the expression level of DUX4 is difficult to measure quantitatively due to its low and sporadic expression, measurement of the expression of DUX4 regulated genes has been shown to correlate with disease activity and progression. Thus, there is a need to identify a DUX4-regulated gene that is readily detected and quantified to serve as a biomarker of disease activity and a prognostic marker of FSHD progression.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY OF THE DISCLOSURE

In one aspect, the present disclosure provides methods for evaluating facioscapulohumeral muscular dystrophy (FSHD) disease activity and by correlation the prognosis of a subject comprising measuring circulating KH homology domain containing 1-like (KHDC1L) protein expression levels in the subject, and evaluating FSHD disease activity of the subject based on the KHDC1L protein expression levels. In some embodiments, the circulating KHDC1L is present in a bodily fluid. In some instances, the bodily fluid is selected from blood serum, blood plasma, blood, lacrimal fluid, synovial fluid, interstitial fluid, saliva, transudate, breast milk, semen, mucus, urine, lymph, and cerebrospinal fluid. In some embodiments, the circulating KHDC1L is detected by a binding agent in the bodily fluid. In some embodiments, the binding agent is an antibody or antigen binding fragment thereof. In some embodiments, the binding agent is an oligonucleotide. In some instances, the oligonucleotide is an aptamer. In some embodiments, the subject has been administered with a therapeutic amount of a compound that decreases human DUX4 mRNA expression levels. In some instances, the compound is selected from a small molecule, a polypeptide, an antibody, a hormone, a lipid, an oligonucleotide, a sugar, a carbohydrate, or a polymer. In some instances, the compound is a binding moiety conjugated to an oligonucleotide. In some embodiments, the disease activity or prognosis of FSHD is determined to be improved if the circulating KHDC1L protein expression level is decreased at least 10%, 20%, 30%, 40%, or more compared to a baseline level within 30, 40, or 50 days after the administration. In some embodiments, the prognosis of FSHD is determined to be improved if the circulating KHDC1L protein expression level is decreased by at least 20%, 30%, or 40% compared to a baseline level and maintained at least 80, 90, or 100 days after the administration.

In another aspect, provided herein is a method for determining an efficacy of a pharmaceutical composition for treating FSHD comprising: measuring a circulating KHDC1L protein expression level in a bodily fluid of a subject after administration of the pharmaceutical composition, and determining the efficacy of the pharmaceutical composition based on the circulating KHDC1L protein expression level. In some embodiments, the bodily fluid is selected from blood serum, blood plasma, blood, lacrimal fluid, synovial fluid, interstitial fluid, saliva, transudate, breast milk, semen, mucus, urine, lymph, and cerebrospinal fluid. In some embodiments, the pharmaceutical composition comprises a compound selected from a small molecule, a protein, an antibody, a hormone, a lipid, an oligonucleotide, a sugar, a carbohydrate, and a polymer. In some instances, the compound is selected from a small molecule, a polypeptide, an antibody, a hormone, a lipid, an oligonucleotide, a sugar, a carbohydrate, or a polymer. In some instances, the compound is a binding moiety conjugated to an oligonucleotide.

In some embodiments, the prognosis of FSHD is determined to be improved if the circulating KHDC1L protein expression level is decreased at least 20%, 30%, or 40% compared to a baseline level within 30, 40, or 50 days after the administration. In some embodiments, the prognosis of FSHD is determined to be improved if the circulating KHDC1L protein expression level is decreased by at least 20%, 30%, or 40% compared to a baseline level and maintained at least 80, 90, or 100 days after the administration.

In another aspect, provided herein is a method of determining an efficacy of a pharmaceutical composition for treating FSHD, comprising: measuring a circulating KHDC1L protein expression level in a bodily fluid of a subject after administration of the pharmaceutical composition; and determining the efficacy of the pharmaceutical composition based on the circulating KHDC1L protein expression level. In some embodiments, the bodily fluid is selected from blood serum, blood plasma, blood, lacrimal fluid, synovial fluid, interstitial fluid, saliva, transudate, breast milk, semen, mucus, urine, lymph, and cerebrospinal fluid. In some embodiments, the pharmaceutical composition comprises a compound selected from a small molecule, a protein, an antibody, a hormone, a lipid, an oligonucleotide, a sugar, a carbohydrate, and a polymer. In some embodiments, the pharmaceutical composition comprises an antibody conjugated to an oligonucleotide. In some embodiments, the efficacy of the pharmaceutical composition is determined to be effective if the circulating KHDC1L protein expression level is decreased by at least 20%, 30%, or 40% compared to a baseline level within 30, 40, or 50 days after the administration. In some embodiments, the efficacy of the pharmaceutical composition is determined to be effective if the circulating KHDC1L protein expression level is decreased by at least 20%, 30%, or 40% compared to a baseline level and maintained at least 80, 90, 100 days after the administration.

In another aspect, provided herein is a method of determining a change in DUX4 expression in a cell comprising measuring KHDC1L protein expression level, wherein the KHDC1L protein is secreted from the cell. In some embodiments, the KHDC1L is present in a cell culture medium which the cell has been cultured in. In some embodiments, the KHDC1L is a circulating protein in a bodily fluid of a subject. In some instances, the bodily fluid is selected from blood serum, blood plasma, blood, urine, lacrimal fluid, synovial fluid, interstitial fluid, saliva, transudate, breast milk, semen, mucus, lymph, and cerebrospinal fluid. In some instances, the KHDC1L protein expression level is measured using a binding agent. In some instances, the binding agent is selected from a small molecule, a polypeptide, a protein, an antibody, a hormone, a lipid, an oligonucleotide, a sugar, a carbohydrate, and a polymer. In some instances, the binding agent is an antibody or antigen binding fragment thereof. In some cases, the antibody is a monoclonal antibody. In some instances, the binding agent is an oligonucleotide. In some cases, the oligonucleotide is an aptamer. In some embodiments, the cell is a muscle cell. In some instances, the muscle cell aberrantly expresses DUX4. In some cases, DUX4 expression is induced by a small molecule. In some instances, wherein the small molecule is doxycycline. In some embodiments, the change in DUX4 expression is correlated with expression levels of KHDC1L proteins.

In another aspect, provided herein is a kit for evaluating a prognosis of FSHD in a subject. In some instances, the kit comprises one or more binding agents that are capable of detecting circulating KHDC1L proteins in a bodily fluid obtained from the subject. In some instances, the one or more binding agents is selected from a small molecule, a polypeptide, a protein, an antibody, a hormone, a lipid, an oligonucleotide, a sugar, a carbohydrate, a polymer, or a combination thereof. In some instances, the one or more binding agents comprises two or more antibodies, two or more aptamers, or an antibody and an aptamer. In some instances, the bodily fluid is selected from blood serum, blood plasma, blood, urine, lacrimal fluid, synovial fluid, interstitial fluid, saliva, transudate, breast milk, semen, mucus, lymph, and cerebrospinal fluid. In some instances, the kit further comprises a sample collection tube. In some instances, the kit further comprises instructions for use.

In another aspect, provided herein is a use of a KHDC1L protein detection reagent in the manufacture of a kit for use in a method for evaluating facioscapulohumeral muscular dystrophy (FSHD) disease activity and prognosis of a subject. In some instances, the method comprises administering a therapeutic amount of a compound that decreases human DUX4 mRNA expression levels, and monitoring concentration of circulating KHDC1L proteins in the subject.

In another aspect, provided herein is a use of a KHDC1L protein detection reagent in the manufacture of a diagnostic kit for use in a method for evaluating a prognosis of FSHD in a subject. In some instances, the method comprises administering a therapeutic amount of a compound that decreases human DUX4 mRNA expression levels, and monitoring concentration of circulating KHDC1L proteins in the subject.

In another aspect, provided herein is a use of a KHDC1L protein detection reagent in the manufacture of a diagnostic kit for use in a method for determining the efficacy of a pharmaceutical composition after a treatment based on a decrease or changes of circulating KHDC1L protein expression levels in a subject. In some instances, the method comprises administering a therapeutic amount of the pharmaceutical composition, and measuring KHDC1L protein expression levels in the subject.

In another aspect, provided herein is a use of a KHDC1L protein detection reagent in the manufacture of a diagnostic kit for use in a method for monitoring drug response of patients with FSHD. In some instances, the method comprises administering a therapeutic amount of a compound that decreases human DUX4 mRNA expression levels, and measuring a reduction of circulating KHDC1L expression levels in the subject.

In another aspect, provided herein is a use of a KHDC1L protein detection reagent in the manufacture of a diagnostic kit for use in a method for determining a change in DUX4 expression by measuring KHDC1L protein expression levels in a subject. In some instances, the method comprises administering a therapeutic amount of a compound that decreases human DUX4 mRNA expression levels, and measuring KHDC1L protein expression levels in the subject.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative aspects, in which the principles of the disclosure are utilized, and the accompanying drawings below.

FIGS. 1A-1B illustrate immunoblot staining of KHDC1L and GAPDH in cell lysates obtained from MB135iDUX4. The cells were treated with (+) or without (−) doxycycline. FIG. 1A illustrates the immunoblot staining of KHDC1L and GAPDH in cell lysates obtained from MB135iDUX4 cells treated with (+) or without (−) doxycycline and recombinant His-tagged KHDC1 or His-tagged KHDC1L detected using a KHDC1L-11-22 monoclonal antibody. FIG. 1B illustrates the immunoblot staining of KHDC1L and GAPDH in cell lysates obtained from MB135iDUX4 cells treated with (+) or without (−) doxycycline and recombinant His-tagged KHDC1 or His-tagged KHDC1L detected using or an anti-KHDC1L-114-128 monoclonal antibody.

FIGS. 2A-2D illustrate the correlation between DUX4 protein activation induced by doxycycline and the detection of KHDC1L protein by an anti-KHDC1L-11-22 monoclonal antibody or an anti-KHDC1L-114-128 monoclonal antibody in MB135iDUX4 cells. FIG. 2A illustrates an immunofluorescence staining of KHDC1L protein detected with an anti-KHDC1L-11-22 monoclonal antibody in MB135iDUX4 cells treated with (+) or without (−) doxycycline. The KHDC1L protein expression is visualized using a TRITC channel and cell nuclei are shown via DAPI staining. FIG. 2B illustrates an immunoblot of KHDC1L protein using an anti-KHDC1L-11-22 monoclonal antibody in MB135iDUX4 cells treated with (+) or without (−) doxycycline after 20 hours, with β-Actin serving as a positive control (a loading control). FIG. 2C illustrates an immunofluorescence staining of KHDC1L protein using an anti-KHDC1L-114-128 monoclonal antibody in MB135iDUX4 cells treated with (+) or without (−) doxycycline. The KHDC1L protein expression is visualized using a TRITC channel and cell nuclei are shown via DAPI staining. FIG. 2D illustrates an immunoblot of KHDC1L protein using an anti-KHDC1L-114-128 monoclonal antibody in MB135iDUX4 cells treated with (+) or without (−) doxycycline after 20 hours, with β-Actin serving as a positive control (a loading control).

FIG. 3 illustrates immunoblot images detecting of DUX4, KHDC1L, GAPDH, and H3XY protein in whole cell (WC) lysates obtained from MB135iDUX4 cells treated with (+) or without (−) doxycycline, or concentrated supernatant obtained from culture media of MB135iDUX4 cells treated with (+) or without (−) doxycycline. KHDC1L protein in both the WC lysates and in the concentrated supernatant from the tissue culture media was detected using an anti-KHDC1L-11-22 monoclonal antibody. GAPDH was detected using anti-GAPDH clone 6C5; H3.XY was detected using rat antisera.

FIG. 4 illustrates immunoblot images detecting KHDC1L, FLOT1, and RPS6 protein in whole cell (WC) lysates obtained from MB135iDUX4 cells treated with (+) or without (−) doxycycline, or concentrated supernatant obtained from culture media of MB135iDUX4 cells treated with (+) or without (−) doxycycline. KHDC1L protein in both the WC lysates and in the concentrated supernatant from the tissue culture media was detected using an anti-KHDC1L-114-128 monoclonal antibody. FLOT1 was detected using anti-FLOT1 clone D2V7J; RPS6 was detected using clone 5G10.

FIG. 5 is a bar graph showing electrochemiluminscent levels of recombinant KHDC1 and KHDC1L proteins coated onto plates with increasing of concentrations (7.8 ng/mL, 31.3 ng/mL, 125 ng/mL, and 500 ng/mL) detected with either anti-KHDC1L monoclonal 11-22 (left) or anti-KHDC1L 114-128 (right) monoclonal antibody conjugated to a SULFO tag at 5 μg/mL using ECLIA. The dotted line denotes baseline level.

FIG. 6A-6B illustrate detection of circulating KHDC1L protein levels in the plasma of FSHD patient or healthy individuals using an electrochemiluminescence immunoassay (ECLIA). FIG. 6A represents a standard curve for the ECLIA assay plotting electrochemiluminescence generated using recombinant KHDC1L at progressively increasing concentrations spiked into 50% human plasma. The standard curve is interpolated to facilitate quantification of endogenous KHDC1L protein levels present in the plasma. FIG. 6B illustrates a graph comparing the levels of circulating KHDC1L protein in plasma samples from FSHD patients (n=20) and healthy individuals (n=20). Both anti-KHDC1L-11-22 and anti-KHDC1L-114-128 monoclonal antibodies were used in the immunoassay to detect KHDC1L protein.

FIGS. 7A-7D illustrate aptamer-mediated detection of KHDC1L protein expression levels in plasma samples of FSHD patient plasma or healthy individuals using a SomaScan 7K platform assay. FIG. 7A illustrates standard curve plotting relative fluorescence units (RFUs) in progressively increasing concentrations of recombinant KHDC1L (rKHDC1L) detected using a DNA aptamer probe, which is then used to assess concentrations of endogenous KHDC1L protein present in plasma samples based on their respective fluorescence intensities. FIG. 7B is a representative graph comparing levels of circulating KHDC1L proteins in plasma samples from FSHD patients (n=20) with in healthy individuals (n=20) using a SomaScan 7K platform assay (**** indicates statistical significance for p<0.0001). FIG. 7C is a graph comparing levels of circulating KHDC1L proteins in plasma samples from FSHD patients (n=98) with healthy individuals (n=100) using a SomaScan 7K platform assay (p<0.0001). FIG. 7D is a graph comparing levels of circulating KHDC1L proteins in plasma samples from FSHD patients (n=98) with healthy individuals (n=100) using a SomaScan 7K platform assay (p<0.0001) with protein concentration represented in log 2 scale.

FIG. 8 is a representative graph illustrating the percentage changes in circulating KHDC1L protein levels in plasma samples from FSHD patients (n=8), following treatment with a 2 mg/kg dose of an antibody-DUX4 siRNA conjugate or a placebo. KHDC1L protein levels were measured at 0 months (baseline), 1.5 months, and 3 months post-treatment timepoints (shown by arrows along the x-axis) or the placebo group (n=4) (PBO) using a SomaScan 7K platform assay. The data shown represent the mean values for both the treatment (n=8) and placebo groups (n=4), with error bars denoting the standard error of the mean (SEM).

FIG. 9 is an exemplary flowchart of a targeted enrichment mass spectrometry assay. Following KHDC1L enrichment using a biotinylated KHDC1L-specific aptamer coupled to streptavidin magnetic beads, mass spectrometry detection of KHDC1L-specific peptides was performed on plasma samples obtained from FSHD patients (Patient #1 and Patient #2). Green shows sequence coverage of the peptide identified in mass spectrometry analysis in each patient. No KHDC1L peptides were detected in plasma from a healthy volunteer.

### DETAILED DESCRIPTION OF THE DISCLOSURE

FSHD is caused by aberrant expression of a gene, DUX4, in skeletal muscle resulting in the inappropriate presence of DUX4 protein. DUX4 functions as a transcription factor that can induce expression of downstream genes that can give rise to the muscle pathology observed in FSHD patients. DUX4-driven gene expression is typically confined to germline, early embryo and early stem cell development. In patients with FSHD, the DUX4 protein in skeletal muscle regulates other gene products, some of which are toxic to the muscle. Thus, evidence of aberrant DUX4-driven gene expression can be regarded as a key molecular signature distinguishing muscle tissue affected by FSHD from healthy muscle. Abnormal expression of DUX4 in FSHD contributes to muscle deterioration and its replacement with fat, resulting in skeletal muscle weakness and progressive disability. Data suggests that reducing expression of the DUX4 gene and its downstream transcriptional program could provide a disease-modifying therapeutic approach for the treatment of FSHD.

The DUX4 gene can be unsilenced, or de-repressed through multiple mechanisms. In FSHD1, which makes up approximately 95% of FSHD cases, mutations can result in the abnormal shortening of the D4Z4, a DNA sequence located near the end of the long arm of chromosome 4, typically characterized by repeats in the sub-telomeric region of the chromosome. Mutated D4Z4 regions contains about 1-10 repeats instead of about 11-100 repeats observed normally, causing DNA hypomethylation of the D4Z4 region and de-repression of DUX4. While patients with FSHD2 are not typically observed to have D4Z4 repeat contraction in the FSHD1 range (10 or fewer D4Z4 units), mutations can occur in a gene that regulates the D4Z4 DNA methylation called SMCHD1. SMCHD1 regulates the establishment of DNA methylation of the D4Z4 units at the DUX4 locus. Pathologic variants in SMCHD1 result in DNA hypomethylation of the D4Z4 region and inappropriate expression of DUX4, triggering the FSHD disease state.

Efforts have been made to develop a therapy or therapeutic compound to treat FSHD by targeting the DUX4 gene and/or modulating aberrant expression of the DUX4 gene. In order to accurately trace the efficacy of such therapy or therapeutical compositions as well as to track the disease activity and prognosis of FSHD in the patients treated with such therapy or therapeutic compositions, quantification of DUX4 modulation is desired.

Yet, even aberrant expression level of DUX4 is lower compared to other mRNA or proteins in the cells or tissues, which renders the quantification of DUX4 regulation challenging in the clinical setting. In some aspects, described herein is a novel biomarker of DUX4 regulation, KHDC1L, that can be detected and quantified from a bodily fluid sample from a patient. It has been observed that levels of circulating KHDC1L protein correlate with DUX4 expression in FSHD patients. In certain aspects, the protein expression level of circulating KHDC1L can be used to determine the efficacy of a pharmaceutical composition or a therapy treating FSHD.

### Pharmaceutical Compositions

Described herein includes a compound or a therapy for treating FSHD. In some aspects, the compound for treating FSHD or a therapy can alleviate one or more symptoms of FSHD, prevent or delay the progress of the FSHD, or reverse the prognosis of the FSHD. The compound can be any form of drug that can be used to treat FSHD. In some embodiments, the compound can be any form of drug that could decrease DUX4 mRNA or protein expression levels and downregulate circulating biomarker protein expression levels in a subject. In some embodiments, the compound decreases DUX4 mRNA or protein expression levels as detected by or evaluated by downregulated circulating biomarker protein expression levels in a subject. In some embodiments, the compound decreases the expression levels of human DUX4 mRNA or protein. In some embodiments, the compound decreases the expression levels of human DUX4 mRNA or protein, thereby treating FSHD. In some embodiments, the compound modulates the expression levels of a circulating biomarker. In some embodiments, the circulating biomarker is associated with muscle dystrophy or atrophy. In some embodiments, the circulating biomarker is KHDC1L. In some embodiments, the compound mediates downregulation of circulating KHDC1L protein expression levels in a bodily fluid of a subject. The bodily fluid includes, but not limited to, blood serum, blood plasma, blood, lacrimal fluid, synovial fluid, interstitial fluid, saliva, transudate, breast milk, semen, mucus, urine, lymph, and cerebrospinal fluid.

Examples of the compound include, but not limited to, a small molecule, amino acids, a peptide, a polypeptide, a protein, an antibody, an antigen, a toxin, a hormone, a lipid, nucleotides, nucleosides, a sugar, a carbohydrate, a polymer, and the like. In some embodiments, the compound is a steroid. In some embodiments, the steroid is a cholesterol, a phospholipid, a di-and triacylglycerol, a fatty acid, and a hydrocarbon. In some embodiments, the compound is an oligonucleotide. In some embodiments, the oligonucleotide is a single stranded oligonucleotide. In some embodiments, the single stranded oligonucleotide is an antisense oligonucleotide (ASO). In some embodiments, the oligonucleotide is a double stranded oligonucleotide. In some embodiments, the double stranded oligonucleotide is a small interfering RNA (siRNA). In some embodiments, the compound is a conjugate. In some embodiments, the compound is an antibody-drug conjugate. In some embodiments, the compound is an antibody-siRNA conjugate. In some embodiments, the compound is a siRNA conjugated to a binding moiety described herein. In some embodiments, the compound is a siRNA-binding moiety conjugate comprising siRNA targeting DUX4 mRNA and a binding moiety described herein. In some embodiments, the therapy comprises a physical therapy or any other therapy that can support muscle strength, prevents deterioration of muscle strength, or slows the progression or reduces severity of FSHD symptoms.

In some embodiments, the compound modulates DUX4 mRNA or protein levels in a muscle cells (e.g., skeletal muscle cells). In some embodiments, the compound modulates DUX4 mRNA or protein levels in a subject. In some embodiments, the compound decreases DUX4 mRNA or protein levels in a muscle cells (e.g., skeletal muscle cells). In some embodiments, the compound decreases DUX4 mRNA or protein levels in a subject. In some embodiments, the compound decreases DUX4 mRNA or protein levels by at least or about 5%, 10%, 20%, 25%, 30%, 40%, 50%, 60%, 70%, 80%, 90%, 95%, or more than 95%. In some embodiments, the compound decreases DUX4 mRNA or protein levels by about 20-50%. In some embodiments, the compound decreases human DUX4 mRNA levels by at least or about 5%, 10%, 20%, 25%, 30%, 40%, 50%, 60%, 70%, 80%, 90%, 95%, or more than 95%. In some embodiments, the compound decreases human DUX4 mRNA levels by about 20-50%. In some embodiments, the compound decreases circulating biomarker protein expression levels by at least or about 5%, 10%, 20%, 25%, 30%, 40%, 50%, 60%, 70%, 80%, 90%, 95%, or more than 95%. In some embodiments, the compound decreases circulating biomarker protein expression levels by about 20-50%. In some embodiments, the compound decreases concentration of circulating biomarker proteins in a subject by at least or about 5%, 10%, 20%, 25%, 30%, 40%, 50%, 60%, 70%, 80%, 90%, 95%, or more than 95%. In some embodiments, the compound decreases circulating biomarker proteins by about 20-50%. In some embodiments, the compound decreases concentration of circulating biomarker proteins in a bodily fluid of a subject by at least or about 5%, 10%, 20%, 25%, 30%, 40%, 50%, 60%, 70%, 80%, 90%, 95%, or more than 95%. In some embodiments, the compound decreases circulating biomarker proteins in a bodily fluid of a subject by about 20-50%. In some embodiments, the compound decrease CK expression levels by at least or about 5%, 1%, 20%, 25%, 30%, 40%, 50%, 60%, 70%, 80%, 90%, 95%, or more than 95%. In some embodiments, the compound decreases CK expression levels by about 20-50%. In some embodiments, the compound decrease concentrations of KHDC1L protein in a subject by at least or about 5%, 10%, 20%, 25%, 30%, 40%, 0%, 60%, 70%, 80%, 90%, 95%, or more than 95%. In some embodiments, the compound decreases concentrations of KHDC1L protein by about 20-50%. In some embodiments, the compound decrease concentrations of KHDC1L protein in a bodily fluid of a subject by at least or about 5%, 10%, 20%, 25%, 30%, 40%, 50%, 60%, 70%, 80%, 90%, 95%, or more than 95%. In some embodiments, the compound decreases concentrations of KHDC1L protein in a bodily fluid of a subject by about 20-50. The bodily fluid includes, but not limited to, blood serum, blood plasma, blood, lacrimal fluid, synovial fluid, interstitial fluid, saliva, transudate, breast milk, semen, mucus, urine, lymph, and cerebrospinal fluid. In some embodiments, the bodily fluid is a bodily fluid sample previously obtained from a subject.

### Binding Agents

In some aspects, the binding agent recognizes or binds KHDC1L protein (e.g., circulating KHDC1L protein). In some instances, the binding agent that recognizes or binds KHDC1L protein (e.g., circulating KHDC1L protein) is a polypeptide. In some instances, the binding agent is an antibody or its fragment thereof. In some instances, the binding agent is a full-length antibody. In some cases, the fragment is an antigen binding fragment. In some instances, the antibody or antigen binding fragment thereof comprises a humanized antibody or antigen binding fragment thereof, murine antibody or antigen binding fragment thereof, chimeric antibody or antigen binding fragment thereof, monoclonal antibody or antigen binding fragment thereof, a binding fragment having a light chain domain and a heavy chain domain, a binding fragment having two light chain domains and two heavy chain domains, a binding fragment having two or more light chain domains and heavy chain domains, monovalent Fab, Fab′, divalent Fab₂, F(ab)′3 fragments, single-chain variable fragment (scFv), bis-scFv, (scFv)₂, diabody, minibody, nanobody, triabody, tetrabody, disulfide stabilized Fv protein (dsFv), single-domain antibody (sdAb), Ig NAR, camelid antibody or antigen binding fragment thereof, bispecific antibody or biding fragment thereof, or a chemically modified derivative thereof. In some instances, the antibody is an anti-KHDC1L antibody binding to the N-terminal domain of the KHDC1L protein. In some instances, the antibody is an anti-KHDC1L antibody binding to the C-terminal domain of the KHDC1L protein.

In some instances, the anti-KHDC1L antibody or antigen binding fragment thereof comprises a variable heavy chain (VH) region and a variable light chain (VL) region, wherein the VH region comprises an HCDR1 sequence comprising a sequence selected from SEQ ID NOs: 17, 23, 29 and 35, an HCDR2 sequence comprising a sequence selected from SEQ ID NOs: 18, 24, 30 and 36, and an HCDR3 sequence comprising a sequence selected from SEQ ID NOs: 19, 25, 31 and 37; and the VJ region comprises an LCDR1 sequence comprising a sequence selected from SEQ ID NOs: 20, 26, 32 and 38, an LCDR2 sequence comprising a sequence selected from the sequences WAS or RMS or SEQ ID NOs: 33 and 39, and an LCDR3 sequence comprising a sequence selected from SEQ ID NOs: 22, 28, 34 and 40.

In some embodiments, the VH region comprises an HCDR1 sequence comprising SEQ ID NO: 17, an HCDR2 sequence comprising SEQ ID NO: 18, and an HCDR3 sequence comprising SEQ ID NO: 19.

In some embodiments, the VL region comprises an LCDR1 sequence comprising SEQ ID NO: 20, an LCDR2 sequence comprising WAS, and an LCDR3 sequence comprising SEQ ID NO: 22.

In some instances, the anti-KHDC1L antibody comprises a VH region and a VL region, in which the VH region comprises an HCDR1 sequence comprising SEQ ID NO: 17, an HCDR2 sequence comprising SEQ ID NO: 18, and an HCDR3 sequence comprising SEQ ID NO: 19; and the VL region comprises an LCDR1 sequence comprising SEQ ID NO: 20, an LCDR2 sequence comprising WAS, and an LCDR3 sequence comprising SEQ ID NO: 22.

In some embodiments, the VH region comprises an HCDR1 sequence comprising SEQ ID NO: 23, an HCDR2 sequence comprising SEQ ID NO: 24, and an HCDR3 sequence comprising SEQ ID NO: 25.

In some embodiments, the VL region comprises an LCDR1 sequence comprising SEQ ID NO: 26, an LCDR2 sequence comprising RMS, and an LCDR3 sequence comprising SEQ ID NO: 28.

In some instances, the anti-KHDC1L antibody comprises a VH region and a VL region, in which the VH region comprises an HCDR1 sequence comprising SEQ ID NO: 23, an HCDR2 sequence comprising SEQ ID NO: 24, and an HCDR3 sequence comprising SEQ ID NO: 25; and the VL region comprises an LCDR1 sequence comprising SEQ ID NO: 26, an LCDR2 sequence comprising RMS, and an LCDR3 sequence comprising SEQ ID NO: 28.

In some embodiments, the VH region comprises an HCDR1 sequence comprising SEQ ID NO: 29, an HCDR2 sequence comprising SEQ ID NO: 30, and an HCDR3 sequence comprising SEQ ID NO: 31.

In some embodiments, the VL region comprises an LCDR1 sequence comprising SEQ ID NO: 32, an LCDR2 sequence comprising SEQ ID NO: 33, and an LCDR3 sequence comprising SEQ ID NO: 34.

In some instances, the anti-KHDC1L antibody comprises a VH region and a VL region, in which the VH region comprises an HCDR1 sequence comprising SEQ ID NO: 29, an HCDR2 sequence comprising SEQ ID NO: 30, and an HCDR3 sequence comprising SEQ ID NO: 31; and the VL region comprises an LCDR1 sequence comprising SEQ ID NO: 32, an LCDR2 sequence comprising SEQ ID NO: 33, and an LCDR3 sequence comprising SEQ ID NO: 34.

In some embodiments, the VH region comprises an HCDR1 sequence comprising SEQ ID NO: 35, an HCDR2 sequence comprising SEQ ID NO: 36, and an HCDR3 sequence comprising SEQ ID NO: 37.

In some embodiments, the VL region comprises an LCDR1 sequence comprising SEQ ID NO: 38, an LCDR2 sequence comprising SEQ ID NO: 39, and an LCDR3 sequence comprising SEQ ID NO: 40.

In some instances, the anti-KHDC1L antibody comprises a VH region and a VL region, in which the VH region comprises an HCDR1 sequence comprising SEQ ID NO: 35, an HCDR2 sequence comprising SEQ ID NO: 36, and an HCDR3 sequence comprising SEQ ID NO: 37; and the VL region comprises an LCDR1 sequence comprising SEQ ID NO: 38, an LCDR2 sequence comprising SEQ ID NO: 39, and an LCDR3 sequence comprising SEQ ID NO: 40.

In some embodiments, the VH region comprises a sequence having at least about 80%, 85%, 90%, 95%, 96% 97%, 98%, 99%, or 100% sequence identity to SEQ ID NO: 3 or 7. In some embodiments, the VL region comprises a sequence having at least about 80%, 85%, 90%, 95%, 96% 97%, 98%, 99%, or 100% sequence identity to SEQ ID NO: 5 or 9.

In some embodiments, the VH region comprises the sequence of SEQ ID NO: 3 or 7.

In some embodiments, the VL region comprises the sequence of SEQ ID NO: 5 or 9.

In some embodiments, the VH region comprises a sequence having at least about 80%, 85%, 90%, 95%, 96% 97%, 98%, 99%, or 100% sequence identity to SEQ ID NO: 3. In some embodiments, the VL region comprises a sequence having at least about 80%, 85%, 90%, 95%, 96% 97%, 98%, 99%, or 100% sequence identity to SEQ ID NO: 5.

In some embodiments, the VH region comprises the sequence of SEQ ID NO: 3 and the VL region comprise the sequence of SEQ ID NO: 5.

In some embodiments, the VH region comprises a sequence having at least about 80%, 85%, 90%, 95%, 96% 97%, 98%, 99%, or 100% sequence identity to SEQ ID NO: 7. In some embodiments, the VL region comprises a sequence having at least about 80%, 85%, 90%, 95%, 96% 97%, 98%, 99%, or 100% sequence identity to SEQ ID NO: 9.

In some embodiments, the VH region comprises the sequence of SEQ ID NO: 7 and the VL region comprise the sequence of SEQ ID NO: 9. some embodiments, the VH region comprises a sequence having at least about 80%, 85%, 90%, 95%, 96% 97%, 98%, 99%, or 100% sequence identity to SEQ ID NO: 4 or 8. In some embodiments, the VL region comprises a sequence having at least about 80%, 85%, 90%, 95%, 96% 97%, 98%, 99%, or 100% sequence identity to SEQ ID NO: 6 or 10.

In some embodiments, the VH region comprises the sequence of SEQ ID NO: 4 or 8.

In some embodiments, the VL region comprises the sequence of SEQ ID NO: 6 or 10.

In some embodiments, the VH region comprises a sequence having at least about 80%, 85%, 90%, 95%, 96% 97%, 98%, 99%, or 100% sequence identity to SEQ ID NO: 4. In some embodiments, the VL region comprises a sequence having at least about 80%, 85%, 90%, 95%, 96% 97%, 98%, 99%, or 100% sequence identity to SEQ ID NO: 6.

In some embodiments, the VH region comprises the sequence of SEQ ID NO: 4 and the VL region comprise the sequence of SEQ ID NO: 6.

In some embodiments, the VH region comprises a sequence having at least about 80%, 85%, 90%, 95%, 96% 97%, 98%, 99%, or 100% sequence identity to SEQ ID NO: 8. In some embodiments, the VL region comprises a sequence having at least about 80%, 85%, 90%, 95%, 96% 97%, 98%, 99%, or 100% sequence identity to SEQ ID NO: 10.

In some embodiments, the VH region comprises the sequence of SEQ ID NO: 8 and the VL region comprise the sequence of SEQ ID NO: 10.

In some aspects, the binding agent that recognizes circulating KHDC1L protein comprises an oligonucleotide. In some instances, the oligonucleotide is an aptamer. In some instances, the aptamer is a DNA aptamer. In some instances, the aptamer is conjugated to an additional binding moiety.

In some aspects, the additional binding moiety that recognizes circulating KHDC1L protein is a plasma protein. In some instances, the plasma protein comprises albumin. In some instances, the binding moiety is albumin. In some instances, albumin is conjugated by one or more of a conjugation chemistry described herein to a polynucleic acid molecule (e.g., aptamer).

In some instances, albumin is conjugated by native ligation chemistry to a polynucleic acid molecule (e.g., aptamer). In some instances, albumin is conjugated by lysine conjugation to a polynucleic acid molecule (e.g., aptamer). In some instances, the additional binding moiety that recognizes circulating KHDC1L protein is a steroid. Exemplary steroids include cholesterol, phospholipids, di-and triacylglycerols, fatty acids, hydrocarbons that are saturated, unsaturated, comprise substitutions, or combinations thereof. In some instances, the steroid is cholesterol. In some instances, the additional binding moiety is cholesterol.

### Methods and Uses

### Biomarkers

Disclosed herein are biomarkers associated with muscle dystrophy or atrophy. In some cases, the biomarker is associated with facioscapulohumeral muscular dystrophy (FSHD). In some cases, the biomarker is associated with DUX4 expression. In some cases, the biomarker is associated with human DUX4 expression. In some cases, the biomarker indicates expression levels of DUX4 mRNA. In some cases, the biomarker indicates expression levels of human DUX4 mRNA. In some aspects, expression of one or more biomarkers that are affected by or downstream of DUX4 expression is also altered or modulated (e.g., decreased) by the decreased expression of human DUX4. For example, in some instances, mRNA expression level of one or more biomarker genes are modulated upon modulation of DUX4 expression. In some instances, protein expression level of one or more biomarker genes are modulated upon modulation of DUX4 expression. In some instances, secretion of one or more biomarker protein or peptide from a cell in a subject (e.g., patient) by modulation of DUX4 expression. In some instances, secretion of one or more biomarker protein or peptide from a cell is affected muscle atrophy by modulation of DUX4 expression. In some instances, secretion of one or more biomarker protein or peptide from a cell affected by FSHD is affected by modulation of DUX4 expression. In some aspects, the markers include mRNAs of, but not limited to, MBD3L2, TRIM43, PRAMEF1, ZSCAN4, KHDC1L, LEUTX, WFDC3, ILVBL, SLC15A2, SORD, and also include proteins that include circulating KHDC1L.

In some cases, the biomarker is a circulating biomarker. In some cases, the circulating biomarker is present in bodily fluid in a subject. In some cases, the biomarker is a circulating peptide or protein. In some cases, a sample of bodily fluid is obtained from a subject or a patient to measure or determine a quantity of a circulating biomarker. Examples of bodily fluid include, not limited to, blood serum, blood plasma, blood, lacrimal fluid, synovial fluid, interstitial fluid, saliva, transudate, breast milk, semen, mucus, urine, lymph, and cerebrospinal fluid. In some embodiments, the circulating biomarker is KHDC1L. In some embodiments, circulating KHDC1L can be obtained and measured from non-invasive samples. In some embodiments, circulating KHDC1L can be obtained and measured from blood samples. In some embodiments, circulating KHDC1L can be obtained and measured from plasma samples. This allows rapid and continuous monitoring of how patients are responding to a therapeutic drug. In some embodiments, circulating KHDC1L is measured before the treatment. In some embodiments, circulating KHDC1L is measured after the treatment. In some embodiments, circulating KHDC1L is measured during the course of the treatment.

In some aspects, the biomarker is associated with diagnosis and/or prognosis of muscle disease, such as FSHD. In some aspects, the biomarker is associated with prognosis of FSHD. In some aspects, a level of circulating KHDC1L is associated with muscle dystrophy or atrophy. In some embodiments, a level of circulating KHDC1L is associated with FSHD. For example, FSHD patients typically have higher levels of circulating KHDC1L compared to healthy individuals. In some aspects, a level, a quantity, or a concentration of circulating KHDC1L can be used to determine a prognosis of FSHD after a treatment with a pharmaceutical composition. In some embodiments, a reduction of circulating KHDC1L expression levels is associated with delayed prognosis of muscle dystrophy or atrophy. In some embodiments, a decreased concentration of circulating KHDC1L proteins is associated with delayed prognosis of muscle dystrophy or atrophy. In some embodiments, a reduction of circulating KHDC1L expression levels is associated with reduction or alleviation of one or more symptoms of FSHD. In some embodiments, a decreased concentration of circulating KHDC1L proteins is associated with reversal of prognosis of FSHD. In some cases, a rapid reduction (e.g., at least 10%, 20%, 30% within 1, 3, 5, 7, 10 days) in circulating KHDC1L expression levels is associated with reduction or alleviation of one or more symptoms, delayed prognosis or reversal or prognosis of FSHD.

### Evaluating a Prognosis of FSHD

In some aspects, described herein is a method of evaluating a prognosis of FSHD in a subject. In some instances, the prognosis of FSHD is evaluated by measuring circulating KH homology domain containing 1-like (KHDC1L) protein expression levels in the subject and evaluating FSHD prognosis of the subject based on the KHDC1L protein expression levels. In some instances, the subject has been administered with a therapeutic amount of a compound as described herein, with a therapeutic amount of a compound that decreases human DUX4 mRNA levels, or received a therapy as described herein. In some instances, the method further comprises a step of administering the subject with the therapeutic amount of a compound that decreases human DUX4 mRNA levels, with a therapeutic amount of a compound as described herein, or providing a therapy to the subject as described herein. In certain instances, circulating KHDC1L protein expression levels are measured at least 10, 20, 30, 40, 50, 60, 70, 80, 90, 100, or more days later, or within 10, 20, 30, 40, 50, 60, 70, 80, 90, 100, or more days after administration of the compound. In some instances, the compound mediates downregulation of circulating KHDC1L protein expression levels. In some embodiments, the circulating KHDC1L is present in a bodily fluid. The bodily fluid includes but not limited to blood serum, blood plasma, blood, lacrimal fluid, synovial fluid, interstitial fluid, saliva, transudate, breast milk, semen, mucus, urine, lymph, and cerebrospinal fluid. In some embodiments, the bodily fluid is selected from blood serum, blood plasma, blood, lacrimal fluid, synovial fluid, interstitial fluid, saliva, transudate, breast milk, semen, mucus, urine, lymph, and cerebrospinal fluid. In some embodiments, the bodily fluid is blood serum. In some embodiments, the bodily fluid is blood plasma. In some embodiments, the bodily fluid is a bodily fluid sample previously obtained from a subject. In some instances, the bodily fluid is processed after being obtained from a subject (e.g., purified, frozen, separation from cells, etc.). Thus, in certain instances, the body fluid is obtained from the subject at least 10, 20, 30, 40, 50, 60, 70, 80, 90, 100, or more days later, or within 10, 20, 30, 40, 50, 60, 70, 80, 90, 100, or more days after administration of the compound, and circulating KHDC1L protein expression levels are measured to determine or evaluate the prognosis of FSHD in the subject based on the KHDC1L protein expression levels or changes of KHDC1L protein expression levels.

In some embodiments, the circulating KHDC1L protein expression level is decreased compared to the baseline (e.g., before the administration, etc.). In some embodiments, the circulating KHDC1L protein expression level is decreased at least about 10%, 20%, 30%, 40%, 50%, 60%, 70%, 80%, 90% or 100% in the FSHD patients administered with the pharmaceutical composition modulating human DUX4 mRNA expression level compared to the baseline. In some embodiments, the circulating KHDC1L protein expression level is decreased at least about 5%, 10%, 15%, 20%, 25%, 30%, 35%, 40%, 45%, 50%, or more in the FSHD patients administered with the pharmaceutical composition modulating human DUX4 mRNA expression level compared to the baseline. In some embodiments, the circulating KHDC1L protein expression level is decreased about 20-50% in the FSHD patients administered with the pharmaceutical composition modulating human DUX4 mRNA expression level compared to the baseline. In some embodiments, the circulating KHDC1L protein expression level is decreased compared to the baseline within 10, 20, 30, 40, 50, 60, 70, 80, 90, 100, or more days after treatment. In some embodiments, the circulating KHDC1L protein expression level is decreased compared to the baseline within 10, 20, 30, 40, 50, or more days after treatment. In some embodiments, the circulating KHDC1L protein expression level is decreased at least 20%, 30%, or 40% compared to the baseline within 30, 40, or 50 days after treatment. In some embodiments, the circulating KHDC1L protein expression level is decreased at least 20%, 30%, or 40% compared to the baseline and maintained at least 80, 90, 100 days after treatment. In some embodiments, the circulating KHDC1L protein expression level is decreased about 20-50% in the FSHD patients administered with the pharmaceutical composition modulating human DUX4 mRNA expression level compared to the baseline and maintained at least 80, 90, 100 days after treatment. In some embodiments, the prognosis of the pharmaceutical composition is determined positive if the circulating KHDC1L protein expression level is decreased. In some embodiments, the prognosis of the pharmaceutical composition is determined positive if the circulating KHDC1L protein expression level is decreased at least about 10%, 20%, 30%, 40%, 50%, 60%, 70%, 80%, 90% or 100% compared to the baseline. In some embodiments, the prognosis of the pharmaceutical composition is determined positive if the circulating KHDC1L protein expression level is decreased at least about 5%, 10%, 15%, 20%, 25%, 30%, 35%, 40%, 45%, 50%, or more compared to the baseline. In some embodiments, the prognosis of the pharmaceutical composition is determined positive if the circulating KHDC1L protein expression level is decreased at least about 20-50% in the FSHD patients treated with the pharmaceutical composition compared to the baseline. In some embodiments, the prognosis of the pharmaceutical composition is determined positive if the circulating KHDC1L protein expression level is decreased compared to the baseline within 10, 20, 30, 40, 50, 60, 70, 80, 90, 100, or more days after treatment. In some embodiments, the prognosis of the pharmaceutical composition is determined positive if the circulating KHDC1L protein expression level is decreased compared to the baseline within 10, 20, 30, 40, 50, or more days after treatment. In some embodiments, the prognosis of the pharmaceutical composition is determined positive if the circulating KHDC1L protein expression level is decreased at least 20%, 30%, or 40% compared to the baseline within 30, 40, or 50 days after treatment. In some embodiments, the prognosis of the pharmaceutical composition is determined positive if the circulating KHDC1L protein expression level is decreased about 20-50% in the FSHD patients treated with the pharmaceutical composition compared to the baseline within 30, 40, or 50 days after treatment. In some embodiments, the prognosis of the pharmaceutical composition is determined positive if the circulating KHDC1L protein expression level is decreased at least 20%, 30%, or 40% compared to the baseline and maintained at least 80, 90, 100 days after treatment. In some embodiments, the prognosis of the pharmaceutical composition is determined positive if the circulating KHDC1L protein expression level is decreased about 20-50% in the FSHD patients treated with the pharmaceutical composition compared to the baseline and maintained at least 80, 90, 100 days after treatment.

### Drug Treatment Efficacy Evaluation

In some aspects, described herein is a method of determining an efficacy of a pharmaceutical composition. In some embodiments, the efficacy of a pharmaceutical composition is determined by measuring circulating KHDC1L protein expression level in a bodily fluid of a subject after administration of the pharmaceutical composition, and determining the efficacy of the pharmaceutical composition after a treatment based on a decreased or changes of circulating KHDC1L protein expression level. In some embodiments, the circulating KHDC1L is present in a bodily fluid. The bodily fluid includes but not limited to blood serum, blood plasma, blood, lacrimal fluid, synovial fluid, interstitial fluid, saliva, transudate, breast milk, semen, mucus, urine, lymph, and cerebrospinal fluid. In some embodiments, the bodily fluid is selected from blood serum, blood plasma, blood, lacrimal fluid, synovial fluid, interstitial fluid, saliva, transudate, breast milk, semen, mucus, urine, lymph, and cerebrospinal fluid. In some embodiments, the bodily fluid is blood serum. In some embodiments, the bodily fluid is blood plasma. In some embodiments, the bodily fluid is a bodily fluid sample previously obtained from a subject. In some instances, the bodily fluid is processed after being obtained from a subject (e.g., purified, frozen, separation from cells, etc.).

In some embodiments, the efficacy of the pharmaceutical composition is determined effective (e.g., effective to treat FSHD) if the circulating KHDC1L protein expression level is decreased. In some embodiments, the efficacy of the pharmaceutical composition is determined effective if the circulating KHDC1L protein expression level is decreased at least about 10%, 20%, 30%, 40%, 50%, 60%, 70%, 80%, 90% or 100% compared to the baseline (e.g., before treatment). In some embodiments, the efficacy of the pharmaceutical composition is determined effective if the circulating KHDC1L protein expression level is decreased at least about 5%, 10%, 15%, 20%, 25%, 30%, 35%, 40%, 45%, 50%, or more compared to the baseline. In some embodiments, the efficacy of the pharmaceutical composition is determined effective if the circulating KHDC1L protein expression level is decreased at least about 20-50% in the FSHD patients treated with the pharmaceutical composition compared to the baseline. In some embodiments, the efficacy of the pharmaceutical composition is determined effective if the circulating KHDC1L protein expression level is decreased compared to the baseline within 10, 20, 30, 40, 50, 60, 70, 80, 90, 100, or more days after treatment. In some embodiments, the efficacy of the pharmaceutical composition is determined effective if the circulating KHDC1L protein expression level is decreased compared to the baseline within 10, 20, 30, 40, 50, or more days after treatment. In some embodiments, the efficacy of the pharmaceutical composition is determined effective if the circulating KHDC1L protein expression level is decreased at least 20%, 30%, or 40% in the FSHD patients treated with the pharmaceutical composition compared to the baseline within 30, 40, or 50 days after treatment. In some embodiments, the efficacy of the pharmaceutical composition is determined effective if the circulating KHDC1L protein expression level is decreased 20-50% in the FSHD patients treated with the pharmaceutical composition compared to the baseline within 30, 40, or 50 days after treatment. In some embodiments, the efficacy of the pharmaceutical composition is determined effective if the circulating KHDC1L protein expression level is decreased at least 20%, 30%, or 40% compared to the baseline and maintained at least 80, 90, 100 days after treatment. In some embodiments, the efficacy of the pharmaceutical composition is determined effective if the circulating KHDC1L protein expression level is decreased about 20-50% compared to the baseline and maintained at least 80, 90, 100 days after treatment.

### Monitoring Drug Response or Efficacy of Drug and or Dosage Regimen

In some aspects, the biomarker can be used to monitor drug response of patients with FSHD. In some cases, a reduction of circulating KHDC1L expression levels is associated with a drug response of FSHD patients. In some aspects, the expression of circulating KHDC1L is decreased at least 10%, 15%, 20%, 25%, 30%, 35%, 40%, 45%, or 50% compared to untreated subject or patient. In some cases, a reduction of circulating KHDC1L expression levels is associated with a drug response of FSHD patients. In some aspects, the expression of circulating KHDC1L is decreased about 20-50% in the FSHD patients treated with a drug modulating DUX4 expression compared to untreated subject or patient. In some aspects, the expression of one or more marker proteins including circulating KHDC1L, as a group or a composite, is decreased at least 10%, 15%, 20%, 25%, 30%, 35%, 40%, 45%, or 50% compared to untreated subject or patient. In some aspects, the expression of one or more marker proteins including circulating KHDC1L, as a group or a composite, is decreased about 20-50% in the FSHD patients treated with a drug modulating DUX4 expression compared to untreated subject or patient. In some cases, a reduction of circulating KHDC1L expression levels is associated with a drug response of FSHD patients. In some aspects, the expression of circulating KHDC1L is decreased at least 10%, 15%, 20%, 25%, 30%, 35%, 40%, 45%, or 50% compared to before treatment in the patient. In some aspects, the expression of circulating KHDC1L is decreased about 20-50% in the FSHD patients treated with a drug modulating DUX4 expression compared to before treatment in the patient. In some aspects, the expression of one or more marker proteins including circulating KHDC1L, as a group or a composite, is decreased at least 10%, 15%, 20%, 25%, 30%, 35%, 40%, 45%, or 50% compared to before treatment in the patient. In some aspects, the expression of one or more marker proteins including circulating KHDC1L, as a group or a composite, is decreased about 20-50% in the FSHD patients treated with a drug modulating DUX4 expression compared to before treatment in the patient.

For example, decreased or increased expression level of the biomarker gene or peptide upon treatment with a drug indicates that the patient is responding to the drug. In some aspects, healthcare practitioners and researchers can evaluate the efficacy of the therapeutic drug based on the level of circulating KHDC1L in the patient. For example, a rapid reduction or maintenance of reduced level of circulating KHDC1L in the patient indicates that the patient is responding well to the treatment of muscle dystrophy or atrophy, such as FSHD. In some instances, maintenance of reduced level of circulating KHDC1L in the patient for at least 3, 5, 7, 10, or 15 days (e.g., within 10% changes, within 5% changes, etc.) indicates that the patient is responding well to the treatment of muscle dystrophy or atrophy, such as FSHD.

In some aspects, the level of circulating KHDC1L in patients can be used to or provide an indicator to adjust dosage regimen. In some cases, healthcare practitioners and researchers can increase or decrease dosage of the drug based on the reduction frequency of, reduction duration of, or absolute or relative level of circulating KHDC1L. In some cases, healthcare practitioners and researchers can increase or decrease dosing interval based on the reduction frequency of, reduction duration of, or absolute or relative level of circulating KHDC1L. This can help improve efficacy and/or decrease toxicity of the treatment of muscle dystrophy or atrophy, such as FSHD.

### Determining a Change in DUX4 Expression

In another aspect, provided herein is a method of determining a change in DUX4 expression by measuring KHDC1L protein expression levels in a fluid or a bodily fluid. In some embodiments, the fluid comprises a cell culture media or cell culture media supernatant. In some instances the fluid is the cell culture media in which the cell has been cultured, and optionally treated with a compound for treating FSHD. In some instances, the fluid comprises one or more secreted molecules from the cells cultured within. In some instances, the fluid comprises one or more secreted peptide or proteins from the cells cultured within. In some embodiments, the bodily fluid is selected from blood serum, blood plasma, blood, lacrimal fluid, synovial fluid, interstitial fluid, saliva, transudate, breast milk, semen, mucus, urine, lymph, and cerebrospinal fluid. In some embodiments, changes of DUX4 expression in the cell, tissues, or a subject can be determined by measuring the circulating KHDC1L protein expression level. In some instances, increase of DUX4 expression (e.g., aberrant increase of DUX4 expression in FSHD) can be indirectly detected or determined by increased circulating KHDC1L protein expression level at least 20%, 30%, 40%, 50%, or more compared to the baseline in a control cell or subject. In another aspect, provided herein is a method of determining a change in DUX4 expression in a subject with a DUX4 mediated disease by measuring KHDC1L protein expression levels in a bodily fluid. The bodily fluid is selected from blood serum, blood plasma, blood, lacrimal fluid, synovial fluid, interstitial fluid, saliva, transudate, breast milk, semen, mucus, urine, lymph, and cerebrospinal fluid. In some embodiments, the increase of DUX4 expression (e.g., aberrant increase of DUX4 expression in DUX4 mediated disease) is determined by increased circulating KHDC1L protein expression level at least 20%, 30%, 40%, 50%, or more compared to the baseline in a healthy subject. In some instances, decrease or inhibition of DUX4 expression (e.g., by effective DUX4 mediated disease treatment) can be indirectly detected or determined by decreased circulating KHDC1L protein expression level at least 20%, 30%, 40%, 50%, or more compared to before treatment.

In another aspect, provided herein is a method of determining a change in DUX4 expression in a subject with FSHD comprising measuring KHDC1L protein expression levels in a bodily fluid. The bodily fluid is selected from blood serum, blood plasma, blood, lacrimal fluid, synovial fluid, interstitial fluid, saliva, transudate, breast milk, semen, mucus, urine, lymph, and cerebrospinal fluid. In some embodiments, the bodily fluid is a bodily fluid sample previously obtained from a subject. In some instances, the bodily fluid is processed after being obtained from a subject (e.g., purified, frozen, separation from cells, etc.). In some embodiments, increase of DUX4 expression (e.g., aberrant increase of DUX4 expression in FSHD) is determined by increased circulating KHDC1L protein expression level at least 20%, 30%, 40%, 50%, or more compared to the baseline in a healthy subject. In some embodiments, increase of DUX4 expression (e.g., aberrant increase of DUX4 expression in FSHD) is determined by increased circulating KHDC1L protein expression level about 20-50% compared to the baseline in a healthy subject. In some instances, decrease or inhibition of DUX4 expression (e.g., by effective FSHD treatment) can be indirectly detected or determined by decreased circulating KHDC1L protein expression level at least 20%, 30%, 40%, 50%, or more compared to before treatment. In some instances, decrease or inhibition of DUX4 expression (e.g., by effective FSHD treatment) can be indirectly detected or determined by decreased circulating KHDC1L protein expression level about 20-50% compared to before treatment.

### Kits/Article of Manufacture

Disclosed herein, in certain aspects, are kits and articles of manufacture for use with one or more of the compositions and methods described herein. In some aspects, provided herein are kits for use in a method for treating facioscapulohumeral muscular dystrophy (FSHD) in a subject. In some aspects, provided herein are kits for use in a method of evaluating a prognosis of FSHD in a subject. In some instances, the prognosis of FSHD is evaluated by measuring circulating KHDC1L protein expression levels in the subject and evaluating FSHD prognosis of the subject based on the KHDC1L protein expression levels. In some aspects, provided herein are kits for use in a method of determining an efficacy of a pharmaceutical composition. In some embodiments, the efficacy of a pharmaceutical composition is determined by measuring circulating KHDC1L protein expression level in a bodily fluid of a subject after administration of the pharmaceutical composition, and determining the efficacy of the pharmaceutical composition after a treatment based on a decreased or changes of circulating KHDC1L protein expression level. In some aspects, provided herein are kits for use in a method of determining a change in DUX4 expression by measuring KHDC1L protein expression levels in a fluid or a bodily fluid.

In some aspects, provided herein is a diagnostic kit comprising one or more binding agents that are capable of detecting circulating KHDC1L proteins in a subject. In some aspects, provided herein is a diagnostic kit comprising one or more binding agents that are capable of measuring concentrations of circulating KHDC1L proteins in a subject. In some aspects, provided herein is a diagnostic kit comprising one or more binding agents that are capable of measuring expression levels of circulating KHDC1L proteins in a subject. In some embodiments, the binding agent is selected from a small molecule, a polypeptide, a protein, an antibody, a hormone, a lipid, an oligonucleotide, a sugar, a carbohydrate, and a polymer. In some embodiments, the binding agent is an antibody or antibody or antigen binding fragment thereof. In some embodiments, the binding agent is an oligonucleotide. In some embodiments, the oligonucleotide is an aptamer. In some embodiments, the kit comprises the same type of the binding agents. In some embodiments, the kit comprises the different types of the binding agents. In some embodiments, the kit comprises two or more antibodies or antigen binding fragments thereof. In some embodiments, the two or more antibodies bind to different regions of KHDC1L. In some embodiments, one of the antibodies binds to amino terminal region of the KHDC1L and the other antibody binds to binding to carboxyl terminal region of the KHDC1L.

In some embodiments, the kit comprises two or more aptamers. In some embodiments, the kit comprises an antibody or antigen binding fragment and an aptamer. In some embodiments, the aptamer is a DNA aptamer.

In some aspects, the kit further comprises a sample collection tube. In some embodiments, the bodily fluid is taken from the subject and stored in the sample collection tube. In some embodiments, the bodily fluid is selected from blood serum, blood plasma, blood, urine, lacrimal fluid, synovial fluid, interstitial fluid, saliva, transudate, breast milk, semen, mucus, lymph, and cerebrospinal fluid. In some embodiments, the bodily fluid is blood.

In some aspects, the kit optionally includes an identifying description or label or instructions relating to its use in the methods described herein. In some embodiments, the kit includes labels listing contents and/or instructions for use, and package inserts with instructions for use. In some embodiments, a label is on or associated with the container. In one embodiment, a label is on a container when letters, numbers or other characters forming the label are attached, molded or etched into the container itself, a label is associated with a container when it is present within a receptacle or carrier that also holds the container, e.g., as a package insert. In one embodiment, a label is used to indicate that the contents are to be used for a specific diagnostic or therapeutic application. The label also indicates directions for use of the contents, such as in the methods described herein.

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The section headings used herein are for organizational purposes only and are not to be construed as limiting the subject matter described.

As used herein, the terms “individual(s)”, “subject(s)” and “patient(s)” mean any mammal. In some aspects, the mammal is a human. In some aspects, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

The term “therapeutically effective amount” relates to an amount of a compound that is sufficient to provide a desired therapeutic effect in a mammalian subject. In some cases, the amount is single or multiple dose administration to a patient (such as a human) for treating, preventing, preventing the onset of, curing, delaying, reducing the severity of, ameliorating at least one symptom of a disorder or recurring disorder, or prolonging the survival of the patient beyond that expected in the absence of such treatment. Naturally, dosage levels of the particular compound employed to provide a therapeutically effective amount vary in dependence of the type of injury, the age, the weight, the gender, the medical condition of the subject, the severity of the condition, the route of administration, and the particular inhibitor employed. In some instances, therapeutically effective amounts of a compound, as described herein, is estimated initially from cell culture and animal models. For example, IC₅₀ values determined in cell culture methods optionally serve as a starting point in animal models, while IC₅₀ values determined in animal models are optionally used to find a therapeutically effective dose in humans.

Skeletal muscle, or voluntary muscle, is generally anchored by tendons to bone and is generally used to effect skeletal movement such as locomotion or in maintaining posture. Although some control of skeletal muscle is generally maintained as an unconscious reflex (e.g., postural muscles or the diaphragm), skeletal muscles react to conscious control. Smooth muscle, or involuntary muscle, is found within the walls of organs and structures such as the esophagus, stomach, intestines, uterus, urethra, and blood vessels.

Skeletal muscle is further divided into two broad types: Type I (or “slow twitch”) and Type II (or “fast twitch”). Type I muscle fibers are dense with capillaries and are rich in mitochondria and myoglobin, which gives Type I muscle tissue a characteristic red color. In some cases, Type I muscle fibers carry more oxygen and sustain aerobic activity using fats or carbohydrates for fuel. Type I muscle fibers contract for long periods of time but with little force. Type II muscle fibers are further subdivided into three major subtypes (IIa, IIx, and IIb) that vary in both contractile speed and force generated. Type II muscle fibers contract quickly and powerfully but fatigue very rapidly, and therefore produce only short, anaerobic bursts of activity before muscle contraction becomes painful.

Unlike skeletal muscle, smooth muscle is not under conscious control.

Cardiac muscle is also an involuntary muscle but more closely resembles skeletal muscle in structure and is found only in the heart. Cardiac and skeletal muscles are striated in that they contain sarcomeres that are packed into highly regular arrangements of bundles. By contrast, the myofibrils of smooth muscle cells are not arranged in sarcomeres and therefore are not striated.

Muscle cells encompass any cells that contribute to muscle tissue. Exemplary muscle cells include myoblasts, satellite cells, myotubes, and myofibril tissues.

As used herein, muscle force is proportional to the cross-sectional area (CSA), and muscle velocity is proportional to muscle fiber length. Thus, comparing the cross-sectional areas and muscle fibers between various kinds of muscles is capable of providing an indication of muscle atrophy. Various methods are known in the art to measure muscle strength and muscle weight, see, for example, “Musculoskeletal assessment: Joint range of motion and manual muscle strength” by Hazel M. Clarkson, published by Lippincott Williams & Wilkins, 2000. The production of tomographic images from selected muscle tissues by computed axial tomography and sonographic evaluation are additional methods of measuring muscle mass.

The term “DUX4 siRNA-conjugate” or “DUX4 siRNA-antibody conjugate” refers to an antibody conjugated to an siRNA hybridizing to a target sequence of the human DUX4 mRNA.

The term “antibody” is used in the broadest sense and covers fully assembled antibodies, antibody fragments that can bind antigen (e.g., Fab, F(ab′)2, Fv, single chain antibodies, VHH, diabodies, antibody chimeras, hybrid antibodies, bispecific antibodies, humanized antibodies, and the like), and recombinant peptides comprising the forgoing.

The terms “antigen binding fragments” and “antibody fragments” are used interchangeably to refer a portion of an intact antibody, preferably the antigen-binding or variable region of the intact antibody. Examples of antibody fragments include Fab, Fab, F(ab′)2, and Fv fragments; VHH; diabodies; linear antibodies (Zapata et al. (1995) Protein Eng. 10:1057-1062); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen.

Depending on the amino acid sequence of the constant domain of their heavy chains, immunoglobulins can be assigned to different classes. There are five major classes of human immunoglobulins: IgA, IgD, IgE, IgG, IgM, and IgY, and several of these may be further divided into subclasses (isotypes), e.g., IgG1, IgG2, IgG3, IgG4, IgA1, and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known. Different isotypes have different effector functions. For example, human IgG1 and IgG3 isotypes have ADCC (antibody dependent cell-mediated cytotoxicity) activity.

In some instances, the CDRs of an antibody is determined according to (i) the Kabat numbering system (Kabat et al. (197) Ann. NY Acad. Sci. 190:382-391 and, Kabat et al. (1991) Sequences of Proteins of Immunological Interest Fifth Edition, U.S. Department of Health and Human Services, NIH Publication No. 91-3242); or (ii) the Chothia numbering scheme, which will be referred to herein as the “Chothia CDRs” (see, e.g., Chothia and Lesk, 1987, J. Mol. Biol., 196:901-917; Al-Lazikani et al., 1997, J. Mol. Biol., 273:927-948; Chothia et al., 1992, J. Mol. Biol., 227:799-817; Tramontano A et al., 1990, J. Mol. Biol. 215(1): 175-82; and U.S. Pat. No. 7,709,226); or (iii) the ImMunoGeneTics (IMGT) numbering system, for example, as described in Lefranc, M.-P., 1999, The Immunologist, 7: 132-136 and Lefranc, M.-P. et al, 1999, Nucleic Acids Res., 27:209-212 (“IMGT CDRs”); or (iv) MacCallum et al, 1996, J. Mol. Biol., 262:732-745. See also, e.g., Martin, A., “Protein Sequence and Structure Analysis of Antibody Variable Domains,” in Antibody Engineering, Kontermann and Diibel, eds., Chapter 31, pp. 422-439, Springer-Verlag, Berlin (2001).

The term “chimeric” antibody refers to an antibody in which a portion of the heavy and/or light chain is derived from a particular source or species, while the remainder of the heavy and/or light chain is derived from a different source or species.

The term “humanized antibody” refers to antibodies in which the framework or the CDRs have been modified to comprise the CDR of an immunoglobulin of different specificity as compared to that of the parent immunoglobulin.

### EXAMPLES

These examples are provided for illustrative purposes only and not to limit the scope of the claims provided herein.

### Example 1: Identification of KHDC1L as a Secreted Protein Induced by DUX4

### Methods

### Preparation of Supernatant

MB135iDUX4 cells (Jagannathan, S. et al. Hum Mol Genet, 25, 4419-4431(2016)) were treated with doxycycline for six hours and subjected to regular growth culture conditions (F10 media supplemented with 20% fetal calf serum and antibiotics (penicillin and streptomycin)) in 15 cm tissue culture plate. The cells were then washed three times in phosphate buffered saline to remove serum proteins and cultured in an additive-free F10 media for additional nine hours. Two parallel plates of cells were treated with doxycycline for biological duplicates, and another two plates of cells were similarly processed without doxycycline treatment for two control biological replicates without DUX4 expression. From a near confluent culture plate, 30 mL of the supernatant was collected and centrifuged at 3260 rcf for 10 minutes at 4 degrees centigrade to remove floating cells. The supernatant was then concentrated in two stages. The supernatant was first filtered through an Amicon Ultra-15 3 kDa MW cut-off filter unit and centrifuged in a swinging bucket rotor at 4000 g to decrease the volume to approximately 200-500 μl. The supernatant was then filtered through an Amicon Ultra-0.5 3 kDa MW cutoff filter and centrifuged for 15 minutes at 14000 g at 4° C. to achieve final volume of approximately 50 μl. Set1 and Set2 denoted in Table 1 below represent biological replicates.

TABLE 1
Predicted secreted proteins enriched in the supernatant of DUX4 expressing MB135
myoblasts compared to MB135 myoblasts not expressing DUX4

| | Set1: | Set2: | GO:0005615 | | Unconventional |
|---|---|---|---|---|---|
| | Abundance | Abundance | extracellular | Conventional | Sec. |
| Gene Name | Ratio (log2) | Ratio (log2) | space | Sec. (SignalP) | (SecretomeP) |
| Ephrin type-A | 2.58* | 2.96 | | | |
| receptor 5 | | | | | |
| (EPHA5) | | | | | |
| Putative KHDC1- | 2.46* | 5.05* | | | ✓ |
| like protein | | | | | |
| (KHDC1L) | | | | | |
| epidermal growth | 2.14* | 4.88* | ✓ | ✓ | |
| factor receptor | | | | | |
| (EGFR) | | | | | |
| Connective tissue | 2.02* | 1.58 | ✓ | ✓ | |
| growth factor | | | | | |
| (CTGF) | | | | | |
| Tumor necrosis | 1.85* | 1.25 | | ✓ | |
| factor receptor | | | | | |
| superfamily | | | | | |
| member 16 | | | | | |
| (NGFR) | | | | | |
| Sodium-dependent | 1.75 | 3.08* | | | |
| phosphate transport | | | | | |
| protein 2B | | | | | |
| (SLC34A2) | | | | | |
| Legumain | 1.59* | 3.19 | ✓ | ✓ | |
| (LGMN) | | | | | |
| Cullin-5 (CUL5) | 1.54 | 2.29 | | | |
| Mannosy1- | 1.53* | 2.53* | ✓ | | |
| oligosaccharide | | | | | |
| 1,2-alpha- | | | | | |
| mannosidase IA | | | | | |
| (MAN1A1) | | | | | |
| N- | 1.38* | 1.7 | ✓ | ✓ | |
| sulphoglucosamine | | | | | |
| sulphohydrolase | | | | | |
| (SGSH) | | | | | |
| Ubiquitin- | 1.32 | 4.89 | | | ✓ |
| conjugating | | | | | |
| enzyme E2 C | | | | | |
| (UBEC2C) | | | | | |
| Small nuclear | 1.31* | 1.51 | ✓ | | |
| ribonucleoprotein- | | | | | |
| associated proteins | | | | | |
| B and B′ (SNRPB) | | | | | |
| HLA class I | 1.28* | 5* | ✓ | ✓ | |
| histocompatibility | | | | | |
| antigen, B-7 alpha | | | | | |
| chain (HLA-B) | | | | | |
| Prosaposin (PSAP) | 1.17* | 1.13 | ✓ | ✓ | |
| Protocadherin-7 | 1.14* | 1.32 | | ✓ | |
| (PCDH7) | | | | | |
| Protocadherin-9 | 1.12* | 1.94 | | ✓ | |
| (PCHD9) | | | | | |

### Results

An analysis of KHDC1L protein expression in the Human Protein Atlas (proteinatlas.org) showed very low RNA expression in all organs with the exception of the testes. Thus, the selection criteria for secreted proteins from DUX4-expressing MB 135 myoblasts included i) proteins with at least a two-fold higher abundance in the doxycycline treated supernatant compared to the control, ii) proteins having a conventional signal peptide (SignalP-5.0 server), or iii) proteins predicted to be secreted by unconventional secretion (SecretomeP-2.0 server). As shown in Table 1, the screening from the cell supernatants identified 16 secreted proteins meeting the selected criteria. Among the selected proteins, KHDC1L fulfilled 2 additional criteria as a potential biomarker for FSHD and other DUX4-mediated diseases: (1) it is a recognized DUX4 target gene with a DUX4 binding site, as determined by chromatin immunoprecipitation (ChIP) near its promoter, that is robustly induced by DUX4 expression in the MB135iDUX4 cells; and (2) it was identified as one of the RNAs that optimally distinguished FSHD-affected muscle biopsies from control muscle biopsies (not affected by FSHD) in a prior RNA sequencing experiment.

Therefore, secreted KHDC1L protein was selected as a potential biomarker for FSHD and other DUX4-mediated diseases and was prioritized for further analysis.

### Example 2: Generation of Two Monoclonal Antibodies to Separate Regions of the KHDC1L Protein

### Methods

To identify and detect KHDC1L protein expression, two mouse monoclonal antibodies binding to two different regions—carboxy terminal region or amino-terminal region of the KHDC1L protein were generated. Antigenic peptides corresponding to the antigenic regions of the amino-terminal region and the carboxy terminal region of the KHDC1L protein were used to generate the antibodies with KHDC1L amino acids 11-22 forming the peptide Ac-[C]EPWWTLPENFHS-amide (amino-terminal region) (SEQ ID NO: 1), and KHDC1L amino acids 114-128 forming the peptide Ac-[C]DDLVTSVSLPPYTGD-OH (carboxyl terminal region) (SEQ ID NO: 2), respectively. The two monoclonal antibodies—i) anti-KHDC1L 11-22 (binding to amino terminal region of the KHDC1L) and ii) anti-KHDC1L 114-128 (binding to carboxyl terminal region of the KHDC1L) were generated by the Fred Hutchinson Cancer Center (FHCC) Antibody Core.

TABLE 2
anti-KHDC1L 11-22 and anti-KHDC1L 114-128 antibodies

| | | | SEQ |
|---|---|---|---|
| | | | ID |
| | Description | Sequence | NO: |
| KHDC1L | Variable Heavy | ATGGGATGGAGCTGTATCATGCTCTTCTTGGCAG | 3 |
| 11-22 | Chain (V_{H}) - | CAACAGCTACAGGTGTCCACTCCCAGGTCCAACT | |
| | nucleic acid | GCAGCAGCCTGGGGCTGAGTTTGTGAAGCCTGGG | |
| | sequence | GCTTCAGTGAAGCTGTCCTGCAAGGCTTCTGACT | |
| | | ACACCTTCACCAACTACTGGATGCACTGGGTGAA | |
| | | ACAGAGGCCTGGACGAGGCCTTGAGTGGATTGGA | |
| | | AGGATTGATCCTAACAGTGGTGGTACTAAGTATA | |
| | | AGGAGAAGTTCAAGAGAAGGGTCACACTGACTG | |
| | | TAGACAAGCCCTCCAGCACAGTCTACATGCAGCT | |
| | | CAACAGCCTGACATCTGAGGACTCTGCGGTCTAT | |
| | | TTCTGTGCAAGAGAAAGGGCTATTTCCTACGGTA | |
| | | GTAGCCCCCTAAATTCTCTGGACTACTGGGGTCA | |
| | | AGGAACCTCAGTCACCGTCTCCTCA | |
| | Variable Heavy | MGWSCIMLFLAATATGVHSQVQLQQPGAEFVKPG | 4 |
| | Chain (V_{H}) - | ASVKLSCKASDYTFTNYWMHWVKQRPGRGLEWIG | |
| | amino acid | RIDPNSGGTKYKEKFKRRVTLTVDKPSSTVYMQL | |
| | sequence | NSLTSEDSAVYFCARERAISYGSSPLNSLDYWGQ | |
| | | GTSVTVSS | |
| | Variable Light | ATGGATTCACAGGCCCAGGTTCTTATGTTACTGCT | 5 |
| | Chain (V_{L}) - | GCTATGGGTATCTGGTACCTGTGGGGACATTGTG | |
| | nucleic acid | ATGTCACAGTCTCCATCCTCCCTAGCTGTGTCAGT | |
| | sequence | TGGAGAGAAGGTTACTATGAGCTGCAAGTCCAGT | |
| | | CAGAGCCTTTTATATAGTAGCAATCAAAAGAACT | |
| | | ACTTGGCCTGGTACCAGCAGAAACCAGGGCAGTC | |
| | | TCCTAAACTGATGATTTACTGGGCATCCACTAGG | |
| | | GAATCTGGGGTCCCTGATCGCTTCACAGGCAGTG | |
| | | GATCTGGGACAGATTTCACTCTCACCATCAGCAG | |
| | | TGTGAAGGCTGAAGACCTGGCAGTTTATTACTGT | |
| | | CAGCAATATTATAGCTATCCGTACACGTTCGGAG | |
| | | GGGGGACCAAGCTGGAAATAAAA | |
| | Variable Light | MDSQAQVLMLLLLWVSGTCGDIVMSQSPSSLAVS | 6 |
| | Chain (V_{L}) - | VGEKVTMSCKSSQSLLYSSNQKNYLAWYQQKPGQ | |
| | amino acid | SPKLMIYWASTRESGVPDRFTGSGSGTDFTLTIS | |
| | sequence | SVKAEDLAVYYCQQYYSYPYTFGGGTKLEIK | |
| KHDC1L | Variable Heavy | ATGGGATGGAGCTGGATCTTTCTCTTTCTCCTGTC | 7 |
| | Chain (V_{H}) - | AGGAACTGCAGGTGTCCTCTCTGAGGTCCAGCTG | |
| | nucleic acid | CAACAATCTGGACCTGAGCTGGTGAAGCCTGGGG | |
| | sequence | CCTCAGTGAAGATATCCTGTAAGGCTTCTGGATA | |
| | | CACGTTCACTGAATACTACATGAACTGGGTGAAG | |
| | | CAGAGCCCTGGAAAGAGCCTTGAGTGGATTGGAG | |
| | | ATATTAATCCTAACAATGGTGGTACTTACTATAAT | |
| | | CAGATGTTCAAGGGCAAGGCCACATTGACTGTAG | |
| | | ACAAGTCCTCCACCACAGCCTACATGGAGCTCCG | |
| | | CAGCCTGACATCTGAGGACTCTGCAGTCTATTAC | |
| | | TGCGCAAGAAAAGTCTCCTCTCCTTACTGGGGCC | |
| | | AAGGGACTCTGGTCACTGTCTCTGCA | |
| 114-128 | Variable Heavy | MGWSWIFLFLLSGTAGVLSEVQLQQSGPELVKPGA | 8 |
| | Chain (V_{H}) - | SVKISCKASGYTFTEYYMNWVKQSPGKSLEWIGDI | |
| | amino acid | NPNNGGTYYNQMFKGKATLTVDKSSTTAYMELR | |
| | sequence | SLTSEDSAVYYCARKVSSPYWGQGTLVTVSA | |
| | Variable Light | ATGAGGTGCCTAGCTGAGTTCCTGGGGCTGCTTG | 9 |
| | Chain (V_{L}) - | TGCTCTGGATCCCTGGAGTCATTGGGGATATTGT | |
| | nucleic acid | GATGACTCAGGCTGCACCCTCTGTATTTGTCACTC | |
| | sequence | CTGGAGAGTCAGTATCCATCTCCTGCAGGTCTAC | |
| | | TAAGAGTCTCCTGCATAGTAATGGCAACACTTAT | |
| | | TTGTATTGGTTCCTGCAGAGGCCAGGCCAGTCTC | |
| | | CTCAGGTCCTGATATATCGGATGTCCAACCTTGCC | |
| | | TCAGGAGTCCCAGACAGGTTCAGTGGCAGTGGGT | |
| | | CAGGAACTGCTTTCACACTGAGAATCAGTAGAGT | |
| | | GGAGGCTGAGGATGTGGGTGTTTATTACTGTATG | |
| | | CAACATCTAGAATATCCTTTCACGTTCGGCTCGG | |
| | | GGACAAAGTTGGAAATAAAA | |
| | Variable Light | MRCLAEFLGLLVLWIPGVIGDIVMTQAAPSVFVTPG | 10 |
| | Chain (V_{L}) - | ESVSISCRSTKSLLHSNGNTYLYWFLQRPGQSPQV | |
| | amino acid | LIYRMSNLASGVPDRFSGSGSGTAFTLRISRVEAED | |
| | sequence | VGVYYCMQHLEYPFTFGSGTKLEIK | |

Underlined sequences in Table 2 are CDR sequences defined by Chothia numbering scheme, and bold sequences in Table 2 are CDR sequences defined by Kabat numbering scheme.

TABLE 2-1
CDR sequences of anti-KHDC1L 11-22 and
anti-KHDC1L 114-128 antibodies
(defined by Chothia)

| | | | SEQ | | | SEQ |
|---|---|---|---|---|---|---|
| | Descrip- | | ID | Descrip- | | ID |
| | tion | Sequence | NO: | tion | Sequence | NO: |
| KHDC1L | HCDR1 | DYTFTNY | 17 | LCDR1 | SQSLLYSSNQKNY | 20 |
| 11-22 | HCDR2 | DPNSGG | 18 | LCDR2 | WAS | |
| | HCDR3 | RAISYGS | 19 | LCDR3 | YYSYPY | 22 |
| | | SPLNSLD | | | | |
| KHDC1L | HCDR1 | GYTFTEY | 23 | LCDR1 | TKSLLHSNGNTY | 26 |
| 114-128 | HCDR2 | INPNNGG | 24 | LCDR2 | RMS | |
| | HCDR3 | VSSP | 25 | LCDR3 | HLEYPF | 28 |

TABLE 2-2
CDR sequences of anti-KHDC1L 11-22 and anti-KHDC1L 114-128 antibodies
(defined by Kabat)

| | | | SEQ | | | SEQ |
|---|---|---|---|---|---|---|
| | | | ID | | | ID |
| | Description | Sequence | NO: | Description | Sequence | NO: |
| KHDC1L | HCDR1 | NYWMH | 29 | LCDR1 | KSSQSLLYSSN | 32 |
| 11-22 | | | | | QKNYLA | |
| | HCDR2 | RIDPNSGGTKYKEKFK | 30 | LCDR2 | WASTRES | 33 |
| | | R | | | | |
| | HCDR3 | ERAISYGSSPLNSLDY | 31 | LCDR3 | QQYYSYPY | 34 |
| KHDC1L | HCDR1 | EYYMN | 35 | LCDR1 | RSTKSLLHSNG | 38 |
| 114-128 | | | | | NTYLY | |
| | HCDR2 | DINPNNGGTYYNQMF | 36 | LCDR2 | RMSNLAS | 39 |
| | | KG | | | | |
| | HCDR3 | KVSSPY | 37 | LCDR3 | MQHLEYPFT | 40 |

### Western Blotting Analysis on Cell Lysates

For the Western blotting analysis, cell lysates were prepared in RIPA buffer with protease and phosphatase inhibitors. 10 μg of protein was mixed with an equal volume of Laemli sample buffer and run on a NuPage 12% Bis-Tris precast gel in NuPage MES SDS Running Buffer, then transferred to a 0.2 μm PVDF membrane in NuPage Transfer Buffer with 20% methanol for 50 min at 30V in an XCell II Blot Module. Membranes were blocked in phosphate buffered saline containing 0.1% Tween-20 and 5% nonfat dry milk before overnight incubation at 4° C. with a primary monoclonal antibody solution at a 1:500 dilution of a 1.63 mg/ml (anti-KHDC1L 11-22) or a 1.71 mg/ml (anti-KHDC1L 114-128).

### Western Blotting on Tissue Culture Supernatant

Near-confluent 15 cm cell culture plates in growth media (F10 supplemented with 20% fetal bovine serum and penicillin/streptomycin) were treated with 1 μg/ml doxycycline for 4 hours and then washed three times in phosphate buffered saline and replacement with F10 without serum supplementation. Another near confluent 15 cm cell culture plates were prepared similarly without doxycycline treatment as a control. The harvested supernatant (25 ml per 15 cm tissue culture plate) was treated with protease/phosphatase inhibitors and then centrifuged and concentrated to a final volume of approximately 50-100 μl as described in Example 1 for the preparation of the samples for mass spectroscopy. Proteins were run on a NuPage pre-cast gel and western immunoblots were performed as described above with characterized antibodies. As a positive control samples prepared from whole cell lysates were run on the same gel.

### Results

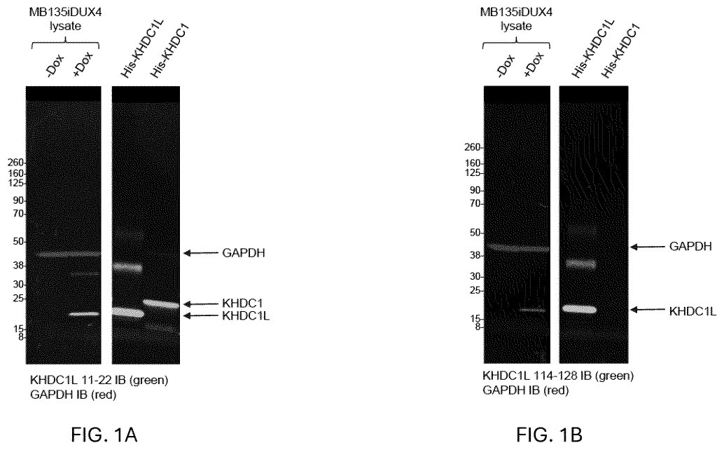

As shown in FIGS. 1A-1B, both KHDC1L monoclonal antibodies (anti-KHDC1L 11-22 and anti-KHDC1L 114-128) detected increased KHDC1L expression upon induction of DUX4 protein expression by doxycycline treatment in MB135iDUX4 cell lysates (lanes 1 and 2, both blots). When tested for specificity against two recombinant family members, KHDC1 and KHDC1L, the anti-KHDC1L 11-22 antibody detected both recombinant His-tagged KHDC1L and His-tagged KHDC1 (FIG. 1A). KHDC1 is a family member of KHDC1L with high sequence homology. However, anti-KHDC1L 114-128 antibody was only observed to be selective for recombinant His-tagged KHDC1L (FIG. 1B).

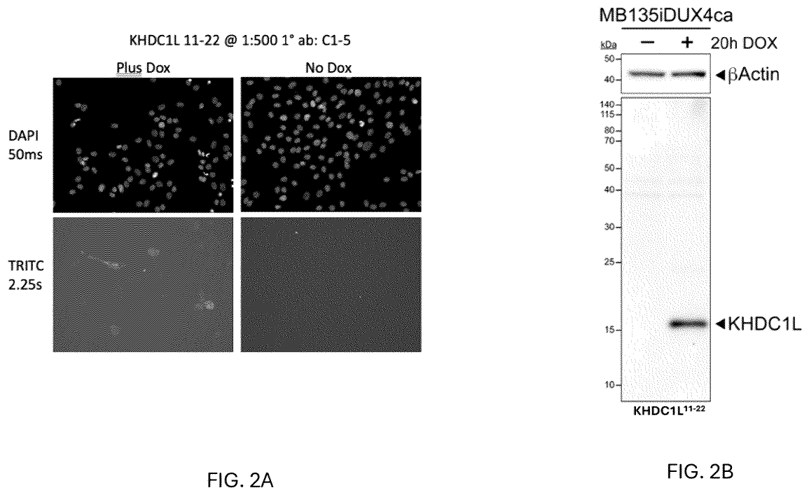

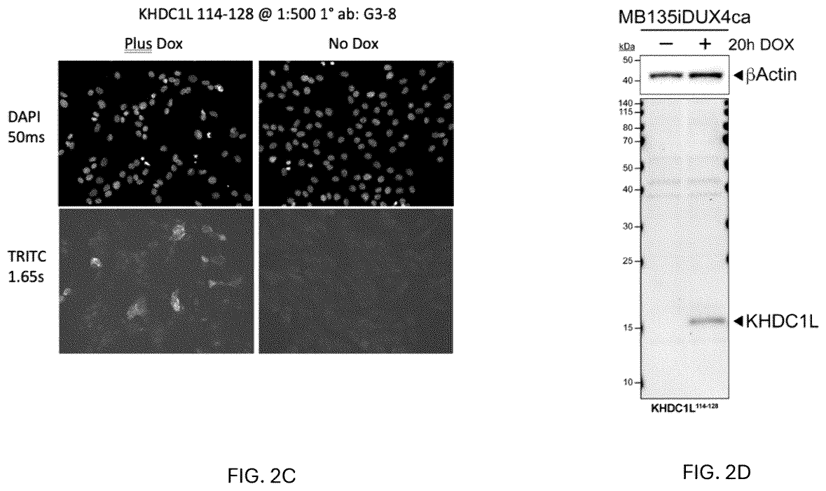

As shown in FIG. 2A and FIG. 2C, subclone C1-5 of anti-KHDC1L 11-22 antibody or subclone G3-8 of anti-KHDC1L 114-128 antibody were used to detect KHDC1L protein by cellular immunofluorescence in MB135iDUX4 cells treated with doxycycline for inducing DUX4 expression. KHDC1L protein expression was not observed in the cells without doxycycline treatment (FIG. 2A and FIG. 2C). In addition, both antibodies identified an immunoreactive protein migrating at the same location and near the predicted size of the KHDC1L protein by Western blotting (FIG. 2B and FIG. 2D). These results indicate that the KHDC1L monoclonal antibodies, subclone CT-5 of anti-KHDC1L 11-22 antibody and subclone G3-8 of anti-KHDC1L 114-128 antibody detected KHDC1L protein expression in cells expressing DUX4 proteins.

### Example 3: Antibody Detection of KHDC1L Protein in Supernatant of Doxycycline-Treated MB135iDUX4 Cells

### Methods

### Western Blotting Analysis on Cell Lysates

For the Western blotting analysis, cell lysates were prepared in RIPA buffer with protease and phosphatase inhibitors. 10 μg of protein was mixed with an equal volume of Laemli sample buffer and run on a NuPage 12% Bis-Tris precast gel in NuPage MES SDS Running Buffer, then transferred to a 0.2 μm PVDF membrane in NuPage Transfer Buffer with 20% methanol for 50 min at 30V in an XCell II Blot Module. Membranes were blocked in phosphate buffered saline containing 0.1% Tween-20 and 5% nonfat dry milk before overnight incubation at 4° C. with a primary monoclonal antibody at a 1:500 dilution of a 1.63 mg/ml (anti-KHDC1L 11-22) or a 1.71 mg/ml (anti-KHDC1L 114-128).

### Western Blotting on Tissue Culture Supernatant

Near-confluent 15 cm cell culture plates in growth media (F10 supplemented with 20% fetal bovine serum and penicillin/streptomycin) were treated with 1 μg/ml doxycycline for 4 hours and then washed three times in phosphate buffered saline and replacement with F10 without serum supplementation. Another near-confluent 15 cm cell culture plate was prepared similarly without doxycycline treatment as a control. The harvested supernatant (25 mL per 15 cm tissue culture plate) was treated with protease/phosphatase inhibitors and then centrifuged and concentrated to a final volume of 50-100 μl in Example 1 to prepare the samples for mass spectroscopy. Proteins were run on a NuPage pre-cast gel and western immunoblots were performed as described above with characterized antibodies. As a positive control samples prepared from whole cell lysates were run on the same gel.

### Results

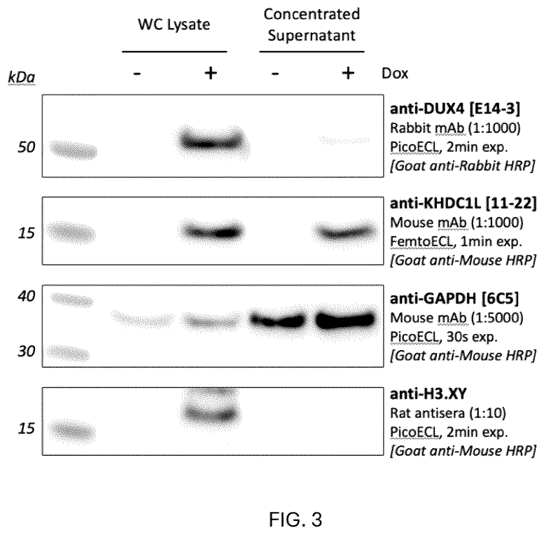

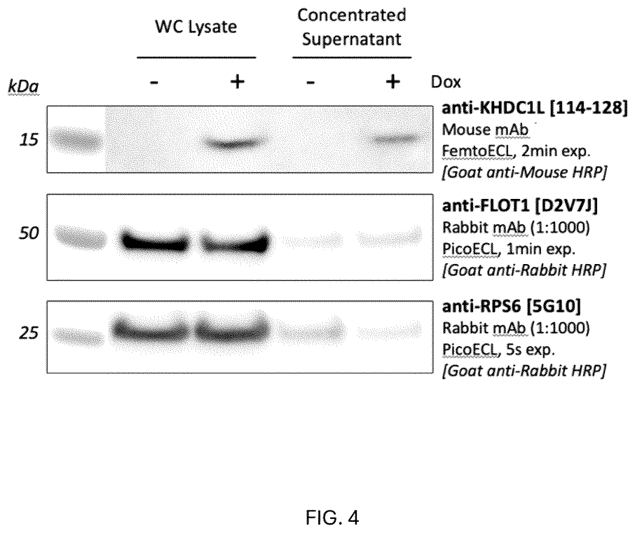

To determine whether induction of DUX4 expression in MB135iDUX4 cells resulted in detectable amounts of KHDC1L protein in the cell culture media, cell culture supernatants from MB135iDUX4 cell culture plates with or without treatment with doxycycline to induce expression of DUX4 expression were concentrated and analyzed by Western blotting. As shown in the immunoblots in FIG. 3 and FIG. 4, the two KHDC1L monoclonal antibodies detected a band signal of ˜16 Kd in size in the supernatant samples that match the size of the KHDC1L protein detected in the cell lysate samples. These results indicate that the detection of DUX4 correlates with the detection of KHDC1L protein in supernatants of cells that were treated with doxycycline.

DUX4 protein expression was detected with a faint band signal in the supernatant of cells treated with doxycycline while KHDC1L protein was more abundantly detected using the anti-KHDC1L 11-22 monoclonal antibody (FIG. 3). As controls, the nuclear proteins H3.X/Y were detected only in the cell extract, while GAPDH was identified both in the cell extract and in the supernatant.

The second KHDC1L antibody, anti-KHDC1L 114-128 antibody, was used to detect KHDC1L protein in cell lysates and supernatant of cells treated with doxycycline (FIG. 4). As controls the cellular scaffolding protein Flot1 and ribosomal protein RPS6 were detected in the cell extract but not in the supernatant (FIG. 4).

Together, these results indicate that the cellular expression of DUX4 protein correlates with the presence of KHDC1L protein in the cell culture supernatant as a secreted protein. Thus, these results suggest that KHDC1L can be used as a circulating biomarker in diseases associated with or related to the aberrant expression of DUX4, e.g., facioscapulohumeral muscular dystrophy (FSHD).

### Example 4: Electrochemiluminescence Immunoassay (ECLIA) Using Anti-KHDC1L 11-22 and Anti-KHDC1L 114-128 Monoclonal Antibodies

### Methods

Plasma samples from healthy volunteers were obtained from BioIVT and plasma samples from FSHD patients were obtained from the Resolve Natural History study. The samples were centrifuged at 1000 g at 4° C. for 15 minutes until plasma and cells were separated. The samples were used according to the standard protocols for ECLIA.

### Results

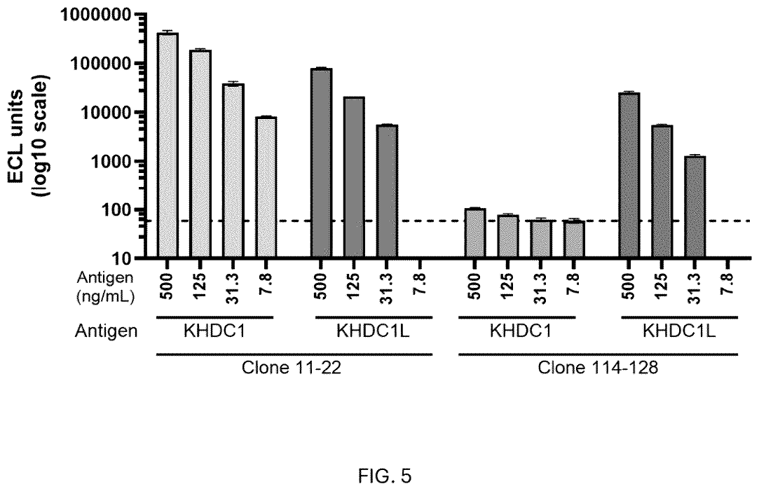

An electrochemiluminescence immunoassay (ECLIA) was conducted using the paired anti-KHDC1L monoclonal antibodies as disclosed herein. Recombinant KHDC1 and KHDC1L were directly coated onto plates and were detected using SULFO-tagged anti-KHDC1L 11-22 and 114-128 monoclonal antibodies using ECLIA as a plate-based detection assay. When tested against recombinant His-tagged KHDC1L and KHDC1 using ECLIA, the anti-KHDC1L 11-22 antibody failed to differentiate between the two family members (FIG. 5). However, the anti-KHDC1L 114-128 antibody was found to be selective for KHDC1L as shown in FIG. 5. In other words, ECLIA assay results showed that anti-KHDC1L 11-22 antibody bound to both KHDC1L and KHDC1 at similar levels, while anti-KHDC1L 114-128 antibody was specific for KHDC1L. Based on these data, an ECLIA assay was designed for analyzing plasma of patients using anti-KHDC1L 114-128 antibody as the capture antibody and anti-KHDC1L 11-22 antibody as the detector antibody.

### Example 5: ECLIA Detection of Increased KHDC1L Levels in FSHD Patient Plasma Samples Compared to Healthy Volunteer Plasma Samples Using Anti-KHDC1L-11-22 and Anti-KHDC1L-114-128 Monoclonal Antibodies

### Methods

Plasma samples from healthy volunteers were obtained from BioIVT and the plasma samples from FSHD patients were obtained from the Resolve Natural History study. The samples were centrifuged at 1000 g at 4° C. for 15 minutes until plasma and cells were separated. The samples were prepared according to standard protocols for ECLIA.

### Results

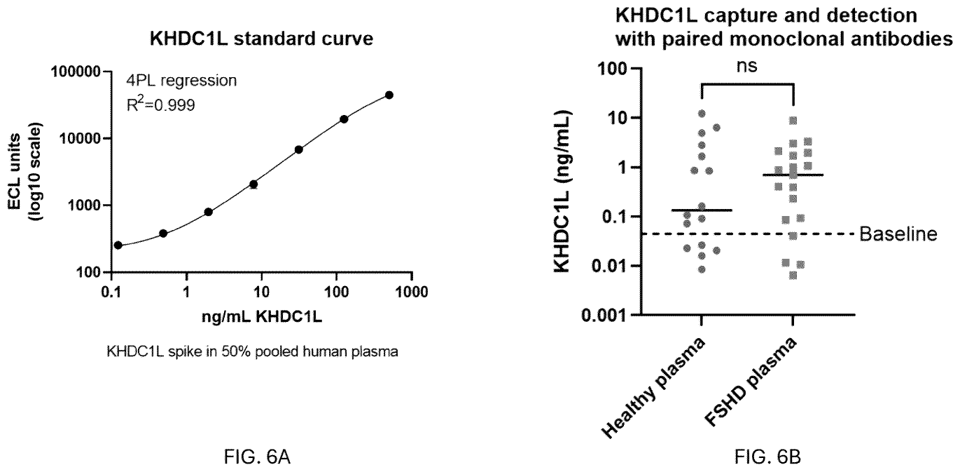

Plasma samples obtained from FSHD patients and healthy volunteers were analyzed by the designed ECLIA assay using the anti-KHDC1L-11-22 and anti-KHDC1L-114-128 monoclonal antibodies (FIGS. 6A-6B). A standard curve for the ECLIA assay was generated using recombinant KHDC1L spiked in 50% human plasma to concentrations of 500, 125, 31.25, 7.81, 1.95, 0.49, 0.12 and 0 ng/mL (FIG. 6A. The standard curve was interpolated to allow quantitation of endogenous KHDC1L levels in clinical samples).

As shown in FIG. 6B, the distribution median of the circulating KHDC1L levels in healthy volunteers was 0.1349 ng/mL (n=16) and the median of KHDC1L in FSHD in patients was 0.6986 ng/mL (n=19). Although the median levels of circulating KHDC1L were trending higher in FSHD patients compared to the levels of healthy volunteers, the results of the assay failed to reach statistical significance (healthy volunteers median=0.1349 ng/mL, n=16; FSHD patients median=0.6986 ng/mL, n=19; p=0.6588).

These results suggest that the ECLIA assay detected higher levels of circulating KHDC1L in plasma of FSHD patients compared to the levels in samples of healthy volunteers, but the assay was not found to be suitable to clinically evaluate changes in circulating KHDC1L in FSHD patient plasma.

### Example 6: Aptamer-Mediated Detection of Increased KHDC1L Levels in FSHD Patient Plasma Compared to Healthy Individual Plasma

### Methods

Plasma samples from healthy volunteers were obtained from BioIVT and plasma samples from FSHD patients were obtained from the Resolve Natural History study. The samples were centrifuged at 1000 g at 4° C. for 15 minutes until plasma and cells were separated.

The plasma samples were prepared according to Somalogic directions for using the SomaScan 7K platform. The experimental procedure followed a sequence of 8 steps: (1) aptamers were synthesized with a fluorophore, photocleavable linker, and biotin; (2) diluted samples were incubated with dilution-specific aptamers bound to streptavidin beads; (3) unbound proteins were washed away, and bound proteins were tagged with biotin; (4) UV light was used to break the photocleavable linker, releasing complexes back into solution; (5) non-specific complexes dissociated while specific complexes remained bound; (6) a polyanionic competitor was added to prevent rebinding of non-specific complexes; (7) biotinylated proteins (and bound aptamers) were captured on new streptavidin beads; and (8) after aptamers were released from the complexes by denaturing the proteins, fluorophores were measured following hybridization to complementary sequences on a microarray chip. The fluorescence intensity detected on the microarray, measured in RFU (Relative Fluorescence Units), is assumed to reflect the amount of available epitope in the original sample (Candia et al., 2022, Nature, Volume 12, pages 17147))

### Results

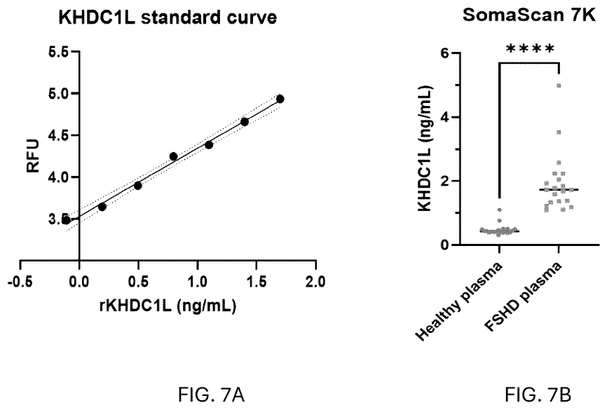

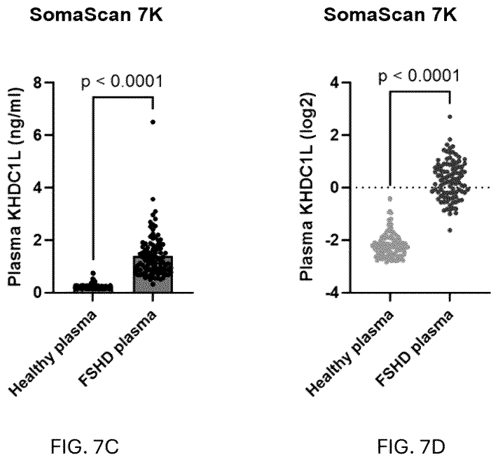

SomaScan 7K platform assay using 2 different specific DNA aptamers designed to bind plasma proteins in a multiplexed format, was used as a detection assay for improved sensitivity to detect circulating KHDC1L protein levels in plasma samples (FIGS. 7A-7D). A standard curve of recombinant KHDC1L peptides was generated to allow absolute quantitation of circulating KHDC1L in the plasma samples (FIG. 7A). As shown in FIG. 7B, the distribution median of the circulating KHDC1L expression levels in healthy volunteers was 0.42987 ng/mL (n=20) and the distribution median of circulating KHDC1L in FSHD patients was 1.730 ng/mL (n=20). The analysis of the SomaScan 7K platform assay results indicate that elevations of circulating KHDC1L levels of in FSHD patient plasma sample were statistically significant compared to circulating KHDC1L levels in healthy volunteer plasma samples (healthy volunteers median=0.42987 ng/mL, n=20; FSHD patients median=1.730 ng/mL, n=20; **** indicates that p<0.0001). This was further investigated in a larger pool of FSHD patient plasma versus age- and sex-matched healthy volunteer plasma. As shown in FIGS. 7C-7D, the mean of the circulating KHDC1L levels in healthy volunteers was 0.2412 ng/mL (n=100) and the mean of the circulating KHDC1L levels in FSHD patients was 1.408 ng/mL (n=98). The analysis of the SomaScan 7K platform assay results indicate statistically significant elevation of circulating KHDC1L levels in FSHD patient plasma sample compared to levels in healthy volunteer plasma (healthy volunteers mean=0.2412 ng/mL, n=100; FSHD plasma mean=1.408 ng/mL, n=98; p<0.0001).

These results indicate that that SomaScan 7K platform assay is clinically suitable to evaluate changes in circulating KHDC1L protein levels in FSHD patient plasma.

### Example 7: Changes in Circulating KHDC1L Protein Levels in FSHD Patients Upon Administration of Therapeutic (Antibody-Dux4 Targeting siRNA Conjugate)

### Methods:

The plasma samples were prepared according to Somalogic directions for performing the SomaScan 7K platform assay as described in Example 6.

### Results:

Although KHDC1L mRNA expression levels have been linked with FSHD in tissue biopsies, a correlation between circulating KHDC1L protein levels in plasma and the prognosis of FSHD treatment has not been previously evaluated experimentally. Circulating KHDC1L protein levels in serum samples from FSHD patients (n=8) administered with a FSHD therapeutic, antibody-Dux4 targeting siRNA conjugate and participants (n=4) from the placebo group (PBO) were measured using the SomaScan 7K platform assay. The individuals in the treatment group were administered an antibody-Dux4 targeting siRNA conjugate at a dose of 2 mg/kg at Day 1, Day 43, and Day 92 (arrows). The antibody-Dux4 targeting siRNA conjugate is a therapeutic targeting aberrant DUX4 expression in cells by siRNA-mediated degradation.

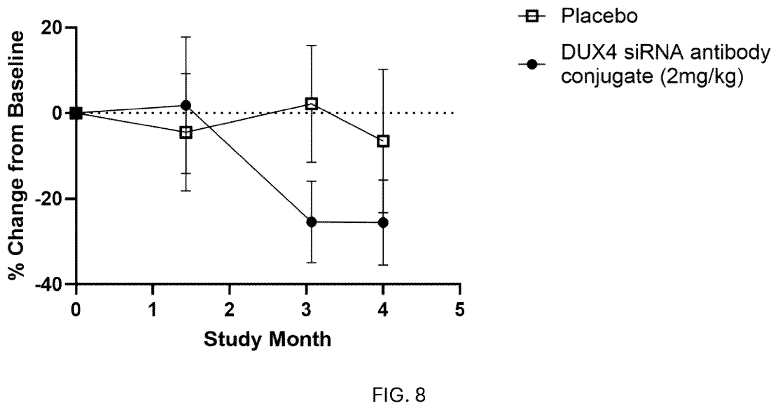

As shown in FIG. 8, individuals in the treatment group that were administered the FSHD therapeutic (an antibody-Dux4 targeting siRNA conjugate) showed a reduction of serum circulating KHDC1L levels following the second dose. After the second dosing, serum KHDC1L levels in these participants decreased to around 25% at Day 92 (month 3) compared to the baseline. After the third dosing, the 25% reduction in serum KHDC1L levels in these individuals was maintained until Day 120 (month 4). On the other hand, KHDC1L concentrations in the Placebo group were relatively stable and did not show any significant changes from the baseline.

Overall, these results indicate that the circulating KHDC1L protein levels in plasma can be reliably used as a biomarker to monitor the disease activity and prognosis of the treatment of FSHD in patients.

### Example 8: Binding Affinities of Mouse Anti-KHDC1L Clones to Biotinylated KHDC1L (KHDC1L-AviTag) by Biolayer Interferometry

### Methods

The binding affinity of anti-KHDC1L clone 114-128 and clone 11-22 to biotinylated KHDC1L (KHDC1L-AviTag) was determined using biolayer interferometry. Anti-KHDC1L clones (4 ug/mL) were immobilized onto anti-mouse IgG Fc biosensor tips and exposed to KHDC1L-AviTag at concentrations ranging from 100 to 3.125 nM. In addition, KHDC1L-AviTag was immobilized onto streptavidin biosensor tips and exposed to anti-KHDC1L clones at concentrations ranging from 50 to 1.5625 nM. Dissociation constants (KD) were subsequently determined.

### Results

Both of the anti-KHDC1L clones tested exhibited strong binding affinity (i.e., in the nanomolar range or lower) with KHDC1L as measured using biolayer interferometry. However, when immobilized to the anti-mouse IgG Fc biosensor tips, anti-KHDC1L clone 114-128 exhibited stronger binding affinity (<50 pM) towards KHDC1L than did clone 11-22 (2.08 nM) (Table 3). Further, when immobilized to the streptavidin biosensor tips, the KHDC1L-AviTag exhibited stronger binding affinity towards clone 11-22 (<50 pM) than towards clone 114-128 (2.40 nM) (Table 3).

TABLE 3
Binding affinities of mouse anti-KHDC1L clones to biotinylated
KHDC1L-AviTag by biolayer interference (BLI)

| | Immobilized | | | | | | |
|---|---|---|---|---|---|---|---|
| Assay # | to tip | Sample | KD (nM) | k_{off} (1/s) | kₒₙ (1/Ms) | Full R\hat{ }2 | Full X\hat{ }2 |
| 1 | Clone 114-128 | KHDC1L-AviTag | OR | OR | 7.81E+04 | 0.9841 | 3.1806 |
| 2 | KHDC1L-AviTag | Clone 114-128 | 2.40 | 1.33E−03 | 5.54E+05 | 0.9878 | 1.9664 |
| 3 | Clone 11-22 | KHDC1L-AivTag | 2.08 | 2.35E−04 | 1.13E+05 | 0.9734 | 3.2683 |
| 4 | KHDC1L-AviTag | Clone 11-22 | OR | OR | 3.45E+04 | 0.9674 | 0.6997 |

*OR = Out of Range of Detection (i.e., <50 pM)

### Example 9: Targeted Enrichment Mass Spectrometry Assay Evaluating KHDC1L Upregulation in FSHD Plasma

### Methods

A biotinylated KHDC1L-specific aptamer was diluted to a 50 nM volume in SB buffer containing 40 mM HEPES (pH 7.5), 100 mM NaCl, 5 mM MgCl₂, 5 mM KCl, 1 mM EDTA, 20 mM NaOH, and 0.05% Tween-20 (pH 7.5). This dilution was then heated to 95° C. for 10 minutes, and then slowly cooled to 37° C. at a rate of 0.1° C. per second. The biotinylated KHDC1L-specific aptamer was coupled to streptavidin magnetic beads, resuspended in a total volume of 100 μL SB buffer, and kept on ice while being protected from light. Pulldowns were performed for 2 hours on plasma from both healthy volunteer patients and FSHD patients (250 μL each). Plasma samples from healthy volunteers and plasma samples from FSHD patients were obtained. The beads were washed three times by rotating in cold HSE buffer containing 20 mM HEPES (pH 7.2), 150 mM NaCl, and 1% Triton X-100, with magnetic capture and removal of buffer after each wash. The captured proteins were eluted in 20 μL denaturing buffer and then heated for 5 minutes at 95° C. The supernatant was collected, reduced and alkylated, and then diluted to a 100 μL volume in buffer containing trypsin for overnight digestion. A 20 μL volume of the digested peptides were loaded onto an Evotip (Evosep 1 system) and injected onto a column for analysis. Data were acquired using a PRM-PASEF targeted acquisition on a timsTOF HT mass spectrometer (Bruker) and analyzed in Skyline. Resulting peptide peaks were manually inspected with a false-discovery rate of <1%.

### Results

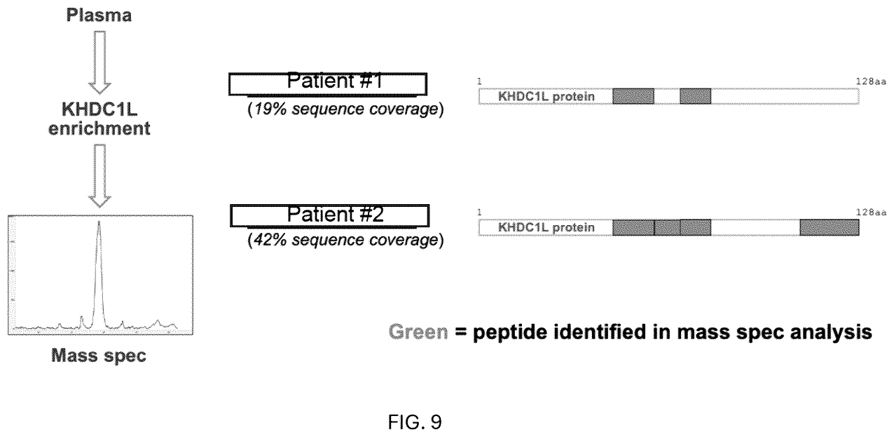

Two unique KHDC1L peptides were detected in FSHD patient plasma #1 and four unique peptides in FSHD patient plasma #2, yielding sequence coverage of 19% and 42%, respectively (FIG. 9). No KHDC1L-specific peptides were identified following enrichment from healthy volunteer plasma, which could represent the lower plasma expression in healthy volunteers compared to disease (Table 4). These results indicate that circulating KHDC1L protein levels in plasma can be used as a biomarker to monitor FSHD in a subject in need thereof.

TABLE 4
KHDC1L peptides detected by mass spectrometry

| | | SEQ | Protein |
|---|---|---|---|
| Plasma | KHDC1L peptides | ID | coverage |
| sample | identified | NO: | (%) |
| Healthy | No peptides | | 0% |
| Volunteer | detected | | |
| FSHD | C[Carbamidomethyl] | 11 | 19% |
| Patient | IELHSHTLIQLER | | |
| #1 | VTVVGPPMAK | 12 | |
| FSHD | C[Carbamidomethyl] | 13 | 42% |
| Patient | IELHSHTLIQLER | | |
| #2 | C[Carbamidomethyl] | 14 | |
| | FTATGQTR | | |
| | VTVVGPPMAK | 15 | |
| | SQPLTNDDLVTSVSLPP | 16 | |
| | YTGD | | |

While preferred aspects of the present disclosure have been shown and described herein, it will be obvious to those skilled in the art that such aspects are provided by way of example only. Numerous variations, changes, and substitutions will now occur to those skilled in the art without departing from the disclosure. It should be understood that various alternatives to the aspects of the disclosure described herein may be employed in practicing the disclosure. It is intended that the following claims define the scope of the disclosure and that methods and structures within the scope of these claims and their equivalents be covered thereby.

What is claimed is:

1. A method of evaluating facioscapulohumeral muscular dystrophy (FSHD) disease activity and prognosis of a subject comprising measuring circulating KH homology domain containing 1-like (KHDC1L) protein expression levels in the subject, and evaluating FSHD prognosis of the subject based on the circulating KHDC1L protein expression levels.

2. The method of claim 1, wherein the circulating KHDC1L is present in a bodily fluid.

3. The method of claim 2, wherein the bodily fluid is selected from blood serum, blood plasma, blood, lacrimal fluid, synovial fluid, interstitial fluid, saliva, transudate, breast milk, semen, mucus, urine, lymph, and cerebrospinal fluid.

4. The method of claim 1, wherein the circulating KHDC1L is detected by a binding agent in the bodily fluid.

5. The method of claim 4, wherein the binding agent is an antibody or antigen binding fragment thereof, an oligonucleotide, or an aptamer.

6. The method of claim 1, wherein the subject has been administered with a therapeutic amount of a compound that decreases human DUX4 mRNA expression levels.

7. The method of claim 6, wherein the compound is selected from a small molecule, a polypeptide, an antibody, a hormone, a lipid, an oligonucleotide, a sugar, a carbohydrate, or a polymer.

8. The method of claim 6, wherein the compound is a binding moiety conjugated to an oligonucleotide.

9. The method of claim 1, wherein the prognosis of FSHD is determined to be improved if the circulating KHDC1L protein expression level is decreased by at least 20%, 30%, or 40% compared to a baseline level within 30, 40, or 50 days after the administration.

10. The method of claim 1, wherein the prognosis of FSHD is determined to be improved if the circulating KHDC1L protein expression level is decreased by at least 20%, 30%, or 40% compared to a baseline level and maintained at least 80, 90, or 100 days after the administration.

11. A method of determining a change in DUX4 expression in a cell comprising measuring KHDC1L protein expression level, wherein the KHDC1L protein is secreted from the cell.

12. The method of claim 11, wherein the KHDC1L is present in a cell culture medium which the cell has been cultured in.

13. The method of claim 11, wherein the KHDC1L is a circulating protein in a bodily fluid of a subject.

14. The method of claim 11, wherein the KHDC1L protein expression level is measured using a binding agent.

15. The method of claim 14, wherein the binding agent is an antibody or antigen binding fragment thereof, an oligonucleotide, or an aptamer.

16. The method of claim 11, wherein the cell is a muscle cell.

17. The method of claim 16, wherein the muscle cell aberrantly expresses DUX4.

18. The method of claim 17, wherein DUX4 expression is induced by a small molecule.

19. The method of claim 18, wherein the small molecule is doxycycline.

20. The method of claim 11, wherein the change in DUX4 expression is correlated with expression levels of KHDC1L proteins.
